(19) United States
(12) Patent Application Publication
(10) Pub. No.: US 2009/0270397 A1
(43) Pub. Date: Oct. 29, 2009

(54) Methods and compositions for the treatment of cancers, such as melanomas and gliomas

(76) Inventors: Seth J. Orlow, New York, NY (US); Ming Chen, Wayne, NJ (US)

Correspondence Address: KLAUBER & JACKSON, 411 HACKENSACK AVENUE, HACKENSACK, NJ, 07601, US

(21) Appl. No.: 12/384,777

(22) Filed: Apr. 8, 2009

Related U.S. Application Data
(60) Provisional application No. 61/123,547, filed on Apr. 8, 2008.

(51) Int. Cl. A61K 31/53; A61K 31/4188; A61K 31/505; A61K 31/517; A61K 31/4985; A61K 31/4965; A61P 35/00

(52) U.S. Cl. 514/245; 514/393; 514/275; 514/266.4; 514/249; 514/255.06

(57) ABSTRACT

A method of potentiating the efficacy of an antineoplastic agent such as temozolomide, comprises the administration of an antifolate agent as defined herein. Also disclosed are compositions, including pharmaceutical compositions, comprising the antineoplastic agent and an antifolate agent, and the use of such compounds and compositions thereof to treat a variety of cancers, such as melanoma. Unit dosage forms and kits are also contemplated and disclosed.

### RELATED APPLICATIONS

The present application claims the benefit under 35 U.S.C. § 119 of U.S. Provisional Application Ser. No. 61/123,547 filed Apr. 8, 2008. The contents of said provisional application is hereby incorporated by reference in its entirety.

### FIELD OF THE INVENTION

The present invention relates to the treatment of cancer, and particularly, to the enhancement of the effectiveness of certain anti-neoplastic agents by administration of an agent that enhances the growth inhibitory effects of those agents.

### BACKGROUND OF THE INVENTION

Several publications and patent documents are referenced in this application in order to more fully describe the state of the art to which this invention pertains. The disclosure of each of these publications and documents is incorporated by reference herein.

Malignant melanoma accounts for about 2% of all cancers in the USA and is increasing in incidence. Most metastatic melanoma patients fail to respond to available therapy, thereby underscoring the need for novel approaches to identify new and more effective treatments. The median survival for stage IV disseminated melanoma is only a few months. The methylating agent dacarbazine (DTIC) is considered the most active drug for the treatment of metastatic melanoma, even though it only exhibits a response rate of 15-20%. Over the past decade, DTIC has been partially replaced by temozolomide (TMZ).

TMZ is structurally related to DTIC. Its oral bioavailability and its ability to cross the blood-brain barrier make it an attractive alternative to DTIC. TMZ exhibits broad-spectrum antitumor activity on diverse cancers, including melanoma, ovarian, colon and brain tumors. In melanoma, TMZ has comparable activity to DTIC. In the US, TMZ is approved for the treatment of certain brain cancers, but used widely to treat melanoma as well. In brain cancers, TMZ resistance is still an important factor, with pediatric brain tumors even more resistant than those of adults.

We hypothesized that there might exist less toxic drugs, approved for other purpose, which might exhibit the unexpected ability to enhance TMZ activity and to overcome resistance to TMZ.

### SUMMARY OF THE INVENTION

Applicants' invention, which is surprising and unobvious in view of the earlier work, is that the chemotherapeutic effects of antineoplastic agents such as temozolomide and dacarbazine can be dramatically potentiated by the administration of an antifolate agent. Thus, human cell cancers which were heretofore insusceptible or only mildly susceptible to these anti neoplastic agents can be more effectively and rapidly treated by the combination of the antineoplastic agent and an antifolate agent.

Accordingly, in a first aspect of the invention, a method for treating cancer comprises the administration of an antineoplastic agent and an antifolate agent, wherein the antifolate agent is capable of crossing the blood-brain barrier. The antifolate agent may be of formula I:

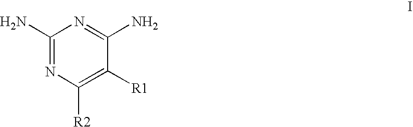

wherein
R¹ is selected from substituted or unsubstituted phenyl;
R² is H, alkyl, substituted alkyl, acyl, substituted acyl, substituted or unsubstituted acylamino, substituted or unsubstituted alkylamino, substituted or unsubstituted alkylthio, substituted or unsubstituted alkoxy, alkoxycarbonyl, substituted alkoxycarbonyl, substituted or unsubstituted alkylarylamino, arylalkyloxy, substituted arylalkyloxy, amino, aryl, substituted aryl, arylalkyl, substituted or unsubstituted sulfoxide, substituted sulfonyl, substituted sulfanyl, substituted or unsubstituted aminosulfonyl, substituted or unsubstituted arylsulfonyl, azido, carboxy, substituted or unsubstituted carbamoyl, cyano, substituted or unsubstituted cycloalkyl, substituted or unsubstituted cycloheteroalkyl, substituted or unsubstituted dialkylamino, halo, heteroaryloxy, substituted or unsubstituted heteroaryl, substituted or unsubstituted heteroalkyl, hydroxy, nitro, and thiol;
or a pharmaceutically acceptable salt thereof;
and stereoisomers and tautomers thereof.

In a particular embodiment, the antifolate agent a lipophilic antifolate agent. In a further particular embodiment, the antifolate agent is selected from pyrimethamine, trimetrexate, Piritrexim, etoprine, metoprine, cycloguanil, methotrexate, trimethoprim, triamterene, amiloride, aminopterin, N,N-dimethylamiloride, N,N-hexamethyleneamiloride, and pterin-6-carboxylic acid. A particular combination comprises temozolomide and pyrimethamine.

Accordingly, it is a principal object of the present invention to provide compositions and methods for improving and extending the therapeutic usefulness of antineoplastic agents such as temozolomide and dacarbazine, by a combination therapy with and antifolate agent.

It is a further object of the invention to provide compositions and methods as aforesaid, wherein the antifolate agents are capable of crossing the blood-brain barrier.

It is a still further object of the invention to provide compositions and methods as aforesaid, wherein the antifolate agents are lipophilic antifolate agents.

It is a yet further object of the present invention to provide therapeutic regimens using these compositions and methods for the optimal potentiation of toxicity of temozolomide to human cancer cells.

It is a still further object of the present invention to provide a repeat dosing regimen of temozolomide which is potentiated by prior or concomitant administration of an antifolate agent.

Other objects and advantages will become apparent to those skilled in the art from a consideration of the ensuing detailed description, taken in conjunction with the following illustrative figures.

### BRIEF DESCRIPTION OF THE DRAWINGS

FIGS. 1A and 1B. Identification of novel compounds that enhance temozolomide (TMZ) chemotherapeutic efficacy in melanoma cells by screening the Spectrum Collection library. FIG. 1A schematically depicts the screening of the Spectrum Collection library to identify novel agents that enhance TMZ efficacy in cultured melanoma cells. Screening was performed with SK-Mel-19 cells in 96-well plates following by MTT assay. Cells in one set of plate were treated with each library compound alone at 1 μM while cells in the parallel plate were treated with a combination of 1 μM library compound and 50 μg/ml TMZ. Positive candidates were later reconfirmed in triplicate. In FIG. 1B, the chemical structures of six compounds that enhanced TMZ activity and induced greater inhibition of cell growth are presented.

FIGS. 2A-2C show that the antifolate agent PYR enhances the chemotherapeutic efficacy of TMZ through inhibition of cell proliferation and survival. Dose-response diagrams of SKM-19 cells exposed to TMZ (FIG. 2A) or pyrimethamine (PYR) (FIG. 2B) alone were prepared for comparison. Melanoma cells were exposed to the indicated concentration of TMZ or PYR for 72 hours. Cell growth inhibition was evaluated by the MTT assay. Survival relative to the control is presented as mean±standard error of the mean of at least three experiments (with viability of control cells set at 100%). In FIG. 2C, PYR enhances TMZ efficacy in various cancer cell lines. Cells were treated with indicated concentrations of TMZ or PYR at the same time and left for 72 hours. Cell viability was determined by MTT assay. SK-MeI 19, SK-Mel 100, SK-Mel 173, and SK-Mel 192 are melanoma cell lines; LN-18 and T98G are glioma cell lines. * P<0.05, **P<0.01.

FIG. 3 is a histogram showing that PYR alters the cell cycle arrest induced by TMZ. Cells were incubated in control medium or medium containing TMZ (25 μg/ml), PYR (0.5 μM), or both for 72 hours prior to harvesting. The harvested cells were stained with propidium iodide and analysed with FACS to determine the proportion of cells in each phase of the cell cycle. This figure is representative of three separate experiments.

FIG. 4 is a gel presenting the results of the administration of an antifolate agent of the invention on the expression of Bcl-2 protein in treated melanoma cells. Cells were incubated in control medium and medium containing drug for 72 hours before harvest. 30 ug total cell lysate was electrophoresed in a 12% SDS-PAGE and transferred to a nitrocellulose membrane. The membrane was blotted with Bcl-2 antibody. Lane 1, control cell; lane 2, DMSO; lane 3, 25 ug/ml TMZ; lane 4, 0.5 uM PYR; lane 5, 25 ug/ml TMZ+0.5 uM PYR.

FIGS. 5A-5B show that PYR increases cell death and apoptosis induced by TMZ in melanoma cells. Melanoma cells were exposed to the indicated concentration of TMZ, PYR or both for 72 hours. In FIG. 5A, cell death was evaluated by trypan blue dye exclusion. * P<0.05, compared to 2 or 3. In FIG. 5B, cleaved Caspase-3 was determined by Western blot analysis. 1. Control; 2. TMZ (25 μg/ml); 3. PYR (0.5 μM), and 4. TMZ and PYR combination.

FIG. 6. TMZ/PYR combination treatment increases DNA damage in melanoma cells. A Western blot is presented showing the increase in H2AX phosphorylation (detected with antibody H2AX-γ) following treatment with TMZ (25 μg/ml) and PYR (0.5 μM) for 2 h in SKM-19 cells. 10 μM CPT treatment is used as a positive control (+). Negative control (−) is cell lysate without any treatment. Levels of actin protein served as loading control.

FIG. 7 is a graph presenting the results of tests of several antifolate agents as enhancers of anti-neoplastic activity. Thus, 0.01 uM of the antifolate agents enhance TMZ efficacy in melanoma cells. Cells were treated with the indicated dose of TMZ alone, or in combination with 0.01 uM PYR, MTX (Methotrexate), TMP (Trimethoprim), TMTX (Trimetrexate), PTX (Piritrexim), and CYC (Cycloguanil). Cell growth was assessed by MTT assay after 72 h drug treatment.

FIGS. 8A-8C. PYR antifolate activity enhances TMZ chemotherapeutic efficacy. A, the growth inhibitory effects of PYR, DDEP, DDMP, CYC, and the classical antifolate MTX on TMZ response curve were examined by MTT assay in melanoma and glioma cells. In SK-MEL 19 cells, 0.005 μM PYR, DDEP, DDMP and 0.001 μM MTX showed significant sensitization to TMZ, while 0.05 μM PYR, DDEP, DDMP and 0.005 μM MTX demonstrated a similar effect in LN-18 cells. * P<0.05,** P<0.01. The cytotoxic effect of the combination of TMZ (25 μg/ml) and PYR (0.5 μM) can be rescued by LV (10 μM) as examined by MTT assay (B) and the level of cleaved PARP (C) in SK-MEL 19 cells. 1. Control; 2. TMZ; 3. PYR; 4. TMZ+PYR; and 5. TMZ+PYR+LV. ** P<0.01, compared to 2, 3, or 5.

FIG. 9 is a further graph presenting the results of tests of several antifolate agents as enhancers of anti-neoplastic activity. In the figure, N1 is pterin-6-carboxylic acid, N2 is aminopterin, N3 is amiloride and N4 is N,N-hexamethylene amiloride. As shown in the figure, each of the tested compounds demonstrated favorable enhancement of the antineoplastic activity of temozolomide.

FIG. 10 presents further data from the testing of additional agents for use in accordance with the invention. Thus, TMZ was administered in combination with compounds S1-S14. The compounds tested are as follows: S1-dipyridamole. S2-methotrexate, S3-minoxidil, S4-prazosin hydrochloride, S5-pyrimethamine, S6-triamterene, S7-trimethoprim, S8-aminopterin, S9-pipemidic acid, S10-piromidic acid, S11-famciclovir, S12-alfuzocin, S13-xanthopterin, and S14-leucopterin.

### DETAILED DESCRIPTION OF THE INVENTION

### Definitions

When describing the compounds, pharmaceutical compositions containing such compounds and methods of using such compounds and compositions, the following terms have the following meanings unless otherwise indicated. It should also be understood that any of the moieties defined forth below may be substituted with a variety of substituents, and that the respective definitions are intended to include such substituted moieties within their scope.

“Acyl” refers to a group or radical —C(O)R²⁰, where R²⁰ is hydrogen, alkyl, cycloalkyl, cycloheteroalkyl, aryl, arylalkyl, heteroalkyl, heteroaryl, heteroarylalkyl as defined herein. Representative examples include, but are not limited to, formyl, acetyl, cylcohexylcarbonyl, cyclohexylmethylcarbonyl, benzoyl, benzylcarbonyl and the like.

“Acylamino” refers to a group or radical —NR²¹C(O)R²², where R²¹ is hydrogen, alkyl, cycloalkyl, cycloheteroalkyl, aryl, arylalkyl, heteroalkyl, heteroaryl, heteroarylalkyl and R²² is hydrogen, alkyl, alkoxy, cycloalkyl, cycloheteroalkyl, aryl, arylalkyl, heteroalkyl, heteroaryl or heteroarylalkyl, as defined herein. Representative examples include, but are not limited to, formylamino, acetylamino, cyclohexylcarbonylamino, cyclohexylmethyl-carbonylamino, benzoylamino, benzylcarbonylamino and the like.

“Acyloxy” refers to the group or radical —OC(O)R²³ where R²³ is hydrogen, alkyl, aryl or cycloalkyl.

“Substituted alkenyl” includes those groups recited in the definition of “substituted” herein, and particularly refers to an alkenyl group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Alkoxy” refers to the group —OR²⁴ where R²⁴ is alkyl. Particular alkoxy groups include, by way of example, methoxy, ethoxy, n-propoxy, isopropoxy, n-butoxy, tert-butoxy, sec-butoxy, n-pentoxy, n-hexoxy, 1,2-dimethylbutoxy, and the like.

“Substituted alkoxy” includes those groups recited in the definition of “substituted” herein, and particularly refers to an alkoxy group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, heteroaryl, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Alkoxycarbonylamino” refers to the group —N R²⁵C(O)R²⁶ where R²⁵ is hydrogen, alkyl, aryl or cycloalkyl, and R²⁶ is alkyl or cycloalkyl.

“Alkyl” refers to monovalent saturated alkane radical groups particularly having up to about 11 carbon atoms, more particularly as a lower alkyl, from 1 to 8 carbon atoms and still more particularly, from 1 to 6 carbon atoms. The hydrocarbon chain may be either straight-chained or branched. This term is exemplified by groups such as methyl, ethyl, n-propyl, isopropyl, n-butyl, iso-butyl, tert-butyl, n-hexyl, n-octyl, tert-octyl and the like. The term “lower alkyl” refers to alkyl groups having 1 to 6 carbon atoms. The term “alkyl” also includes “cycloalkyls” as defined below.

“Substituted alkyl” includes those groups recited in the definition of “substituted” herein, and particularly refers to an alkyl group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, heteroaryl, keto, nitro, thioalkoxy, substituted thioalkoxy, thioaryloxy, thioketo, thiol, alkyl-S(O)—, aryl-S(O)—, alkyl-S(O)₂—, and aryl-S(O)₂—.

“Alkylene” refers to divalent saturated alkene radical groups having 1 to 11 carbon atoms and more particularly 1 to 6 carbon atoms which can be straight-chained or branched. This term is exemplified by groups such as methylene (—CH₂—), ethylene (—CH₂CH₂—), the propylene isomers (e.g., —CH₂CH₂CH₂— and —CH(CH₃)CH₂—) and the like.

“Substituted alkylene” includes those groups recited in the definition of “substituted” herein, and particularly refers to an alkylene group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Alkenyl” refers to monovalent olefinically unsaturated hydrocarbyl groups preferably having 2 to 11 carbon atoms, particularly, from 2 to 8 carbon atoms, and more particularly, from 2 to 6 carbon atoms, which can be straight-chained or branched and having at least 1 and particularly from 1 to 2 sites of olefinic unsaturation. Particular alkenyl groups include ethenyl (—CH═CH₂), n-propenyl (—CH₂CH═CH₂), isopropenyl (—C(CH₃)═CH₂), vinyl and substituted vinyl, and the like.

“Alkenylene” refers to divalent olefinically unsaturated hydrocarbyl groups particularly having up to about 11 carbon atoms and more particularly 2 to 6 carbon atoms which can be straight-chained or branched and having at least 1 and particularly from 1 to 2 sites of olefinic unsaturation. This term is exemplified by groups such as ethenylene (—CH═CH—), the propenylene isomers (e.g., —CH═CHCH₂— and —C(CH₃)═CH— and —CH═C(CH₃)—) and the like.

“Alkynyl” refers to acetylenically or alkynically unsaturated hydrocarbyl groups particularly having 2 to 11 carbon atoms and more particularly 2 to 6 carbon atoms which can be straight-chained or branched and having at least 1 and particularly from 1 to 2 sites of alkynyl unsaturation. Particular non-limiting examples of alkynyl groups include acetylenic, ethynyl (—C≡CH), propargyl (—CH₂C≡CH), and the like.

“Substituted alkynyl” includes those groups recited in the definition of “substituted” herein, and particularly refers to an alkynyl group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Alkanoyl” or “acyl” as used herein refers to the group R²⁷—C(O)—, where R²⁷ is hydrogen or alkyl as defined above.

“Aryl” refers to a monovalent aromatic hydrocarbon group derived by the removal of one hydrogen atom from a single carbon atom of a parent aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. Particularly, an aryl group comprises from 6 to 14 carbon atoms.

“Substituted Aryl” includes those groups recited in the definition of “substituted” herein, and particularly refers to an aryl group that may optionally be substituted with 1 or more substituents, for instance from 1 to 5 substituents, particularly 1 to 3 substituents, selected from the group consisting of acyl, acylamino, acyloxy, alkenyl, substituted alkenyl, alkoxy, substituted alkoxy, alkoxycarbonyl, alkyl, substituted alkyl, alkynyl, substituted alkynyl, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, nitro, thioalkoxy, substituted thioalkoxy, thioaryloxy, thiol, alkyl-S(O)—, aryl-S(O)—, alkyl-S(O)₂— and aryl-S(O)₂—.

“Fused Aryl” refers to an aryl having two of its ring carbon in common with a second aryl ring or with an aliphatic ring.

“Alkaryl” refers to an aryl group, as defined above, substituted with one or more alkyl groups, as defined above.

“Aralkyl” or “arylalkyl” refers to an alkyl group, as defined above, substituted with one or more aryl groups, as defined above.

“Aryloxy” refers to —O-aryl groups wherein “aryl” is as defined above.

“Alkylamino” refers to the group alkyl-NR²⁸R²⁹, wherein each of R²⁸ and R²⁹ are independently selected from hydrogen and alkyl.

“Arylamino” refers to the group aryl-NR³⁰R³¹, wherein each of R³⁰ and R³¹ are independently selected from hydrogen, aryl and heteroaryl.

“Alkoxyamino” refers to a radical —N(H)OR³² where R³² represents an alkyl or cycloalkyl group as defined herein.

“Alkoxycarbonyl” refers to a radical —C(O)-alkoxy where alkoxy is as defined herein.

“Alkylarylamino” refers to a radical —NR³³R³⁴ where R³³ represents an alkyl or cycloalkyl group and R³⁴ is an aryl as defined herein.

“Alkylsulfonyl” refers to a radical —S(O)₂R³⁵ where R³⁵ is an alkyl or cycloalkyl group as defined herein. Representative examples include, but are not limited to, methylsulfonyl, ethylsulfonyl, propylsulfonyl, butylsulfonyl and the like.

“Alkylsulfinyl” refers to a radical —S(O)R³⁵ where R³⁵ is an alkyl or cycloalkyl group as defined herein. Representative examples include, but are not limited to, methylsulfinyl, ethylsulfinyl, propylsulfinyl, butylsulfinyl and the like.

“Alkylthio” refers to a radical —SR³⁵ where R³⁵ is an alkyl or cycloalkyl group as defined herein that may be optionally substituted as defined herein. Representative examples include, but are not limited to, methylthio, ethylthio, propylthio, butylthio, and the like.

“Amino” refers to the radical —NH₂.

“Substituted amino” includes those groups recited in the definition of “substituted” herein, and particularly refers to the group —N(R³⁶)₂ where each R³⁶ is independently selected from the group consisting of hydrogen, alkyl, substituted alkyl, alkenyl, substituted alkenyl, alkynyl, substituted alkynyl, aryl, cycloalkyl, substituted cycloalkyl, and where both R groups are joined to form an alkylene group. When both R groups are hydrogen, —N(R³⁶)₂ is an amino group.

“Aminocarbonyl” refers to the group —C(O)NR³⁷R³⁷ where each R³⁷ is independently hydrogen, alkyl, aryl and cycloalkyl, or where the R³⁷ groups are joined to form an alkylene group.

“Aminocarbonylamino” refers to the group —NR³⁸C(O)NR³⁸R³⁸ where each R³⁸ is independently hydrogen, alkyl, aryl or cycloalkyl, or where two R groups are joined to form an alkylene group.

“Aminocarbonyloxy” refers to the group —OC(O)NR³⁹R³⁹ where each R³⁹ is independently hydrogen, alkyl, aryl or cycloalkyl, or where the R groups are joined to form an alkylene group.

“Arylalkyloxy” refers to an —O-arylalkyl radical where arylalkyl is as defined herein.

“Arylamino” means a radical —NHR⁴⁰ where R⁴⁰ represents an aryl group as defined herein.

“Aryloxycarbonyl” refers to a radical —C(O)—O-aryl where aryl is as defined herein.

“Arylsulfonyl” refers to a radical —S(O)₂R⁴¹ where R⁴¹ is an aryl or heteroaryl group as defined herein.

“Azido” refers to the radical —N₃.

“Bicycloaryl” refers to a monovalent aromatic hydrocarbon group derived by the removal of one hydrogen atom from a single carbon atom of a parent bicycloaromatic ring system. Typical bicycloaryl groups include, but are not limited to, groups derived from indane, indene, naphthalene, tetrahydronaphthalene, and the like. Particularly, an aryl group comprises from 8 to 11 carbon atoms.

“Bicycloheteroaryl” refers to a monovalent bicycloheteroaromatic group derived by the removal of one hydrogen atom from a single atom of a parent bicycloheteroaromatic ring system. Typical bicycloheteroaryl groups include, but are not limited to, groups derived from benzofuran, benzimidazole, benzindazole, benzdioxane, chromene, chromane, cinnoline, phthalazine, indole, indoline, indolizine, isobenzofuran, isochromene, isoindole, isoindoline, isoquinoline, benzothiazole, benzoxazole, naphthyridine, benzoxadiazole, pteridine, purine, benzopyran, benzpyrazine, pyridopyrimidine, quinazoline, quinoline, quinolizine, quinoxaline, benzomorphan, tetrahydroisoquinoline, tetrahydroquinoline, and the like. Preferably, the bicycloheteroaryl group is between 9-11 membered bicycloheteroaryl, with 5-10 membered heteroaryl being particularly preferred. Particular bicycloheteroaryl groups are those derived from benzothiophene, benzofuran, benzothiazole, indole, quinoline, isoquinoline, benzimidazole, benzoxazole and benzdioxane.

“Carbamoyl” refers to the radical —C(O)N(R⁴²)₂ where each R⁴² group is independently hydrogen, alkyl, cycloalkyl or aryl, as defined herein, which may be optionally substituted as defined herein.

“Carboxy” refers to the radical —C(O)OH.

“Carboxyamino” refers to the radical —N(H)C(O)OH.

“Cycloalkyl” refers to cyclic hydrocarbyl groups having from 3 to about 10 carbon atoms and having a single cyclic ring or multiple condensed rings, including fused and bridged ring systems, which optionally can be substituted with from 1 to 3 alkyl groups. Such cycloalkyl groups include, by way of example, single ring structures such as cyclopropyl, cyclobutyl, cyclopentyl, cyclooctyl, 1-methylcyclopropyl, 2-methylcyclopentyl, 2-methylcyclooctyl, and the like, and multiple ring structures such as adamantanyl, and the like.

“Substituted cycloalkyl” includes those groups recited in the definition of “substituted” herein, and particularly refers to a cycloalkyl group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Cycloalkoxy” refers to the group —OR⁴³ where R⁴³ is cycloalkyl. Such cycloalkoxy groups include, by way of example, cyclopentoxy, cyclohexoxy and the like.

“Cycloalkenyl” refers to cyclic hydrocarbyl groups having from 3 to 10 carbon atoms and having a single cyclic ring or multiple condensed rings, including fused and bridged ring systems and having at least one and particularly from 1 to 2 sites of olefinic unsaturation. Such cycloalkenyl groups include, by way of example, single ring structures such as cyclohexenyl, cyclopentenyl, cyclopropenyl, and the like.

“Substituted cycloalkenyl” includes those groups recited in the definition of “substituted” herein, and particularly refers to a cycloalkenyl group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Fused Cycloalkenyl” refers to a cycloalkenyl having two of its ring carbon atoms in common with a second aliphatic or aromatic ring and having its olefinic unsaturation located to impart aromaticity to the cycloalkenyl ring.

“Cyanato” refers to the radical —OCN.

“Cyano” refers to the radical —CN.

“Dialkylamino” means a radical —NR⁴⁴R⁴⁵ where R⁴⁴ and R⁴⁵ independently represent an alkyl, substituted alkyl, aryl, substituted aryl, cycloalkyl, substituted cycloalkyl, cycloheteroalkyl, substituted cycloheteroalkyl, heteroaryl, or substituted heteroaryl group as defined herein.

“Ethenyl” refers to substituted or unsubstituted —(C═C)—.

“Ethylene” refers to substituted or unsubstituted —(C—C)—.

“Ethynyl” refers to —(C≡C)—.

“Halo” or “halogen” refers to fluoro, chloro, bromo and iodo. Preferred halo groups are either fluoro or chloro.

“Hydroxy” refers to the radical —OH.

“Nitro” refers to the radical —NO₂.

“Substituted” refers to a group in which one or more hydrogen atoms are each independently replaced with the same or different substituent(s). Typical substituents include, but are not limited to, —X, —R⁴⁶, —O⁻, ═O, —OR⁴⁶, —SR⁴⁶, —S⁻, —S⁻, —NR⁴⁶R⁴⁷, ═NR⁴⁶, —CX₃, —CF₃, —CN, —OCN, —SCN, —NO, —NO₂, ═N₂, —N₃, —S(O)₂O⁻, —S(O)₂OH, —S(O)₂R⁴⁶, —OS(O₂)O⁻, —OS(O)₂R⁴⁶, —P(O)(O⁻)₂, —P(O)(OR⁴⁶)(O⁻), —OP(O)(OR⁴⁶)(OR⁴⁷), —C(O)R⁴⁶, —C(S)R⁴⁶, —C(O)OR⁴⁶, —C(O)NR⁴⁶R⁴⁷, —C(O)O⁻, —C(S)OR⁴⁶, —NR⁴⁸C(O)NR⁴⁶R⁴⁷, —NR⁴⁸C(S)NR⁴⁶R⁴⁷, —NR⁴⁹C(NR⁴⁸)NR⁴⁶R⁴⁷ and —C(NR⁴⁸)NR⁴⁶R⁴⁷, where each X is independently a halogen; each R⁴⁶, R⁴⁷, R⁴⁸ and R⁴⁹ are independently hydrogen, alkyl, substituted alkyl, aryl, substituted alkyl, arylalkyl, substituted alkyl, cycloalkyl, substituted alkyl, cycloheteroalkyl, substituted cycloheteroalkyl, heteroalkyl, substituted heteroalkyl, heteroaryl, substituted heteroaryl, heteroarylalkyl, substituted heteroarylalkyl, —NR⁵⁰R⁵¹, —C(O)R⁵⁰ or —S(O)₂R⁵⁰ or optionally R⁵⁰ and R⁵¹ together with the atom to which they are both attached form a cycloheteroalkyl or substituted cycloheteroalkyl ring; and R⁵⁰ and R⁵¹ are independently hydrogen, alkyl, substituted alkyl, aryl, substituted alkyl, arylalkyl, substituted alkyl, cycloalkyl, substituted alkyl, cycloheteroalkyl, substituted cycloheteroalkyl, heteroalkyl, substituted heteroalkyl, heteroaryl, substituted heteroaryl, heteroarylalkyl or substituted heteroarylalkyl.

Examples of representative substituted aryls include the following

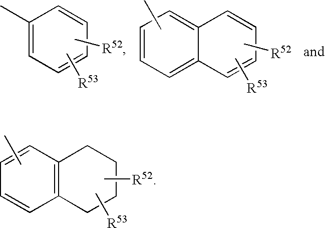

In these formulae one of R⁵² and R⁵³ may be hydrogen and at least one of R⁵² and R⁵³ is each independently selected from alkyl, alkenyl, alkynyl, cycloheteroalkyl, alkanoyl, alkoxy, aryloxy, heteroaryloxy, alkylamino, arylamino, heteroarylamino, NR⁵⁴COR⁵⁵, NR⁵⁴SOR⁵⁵, NR⁵⁴SO₂R⁵⁷, COOalkyl, COOaryl, CONR⁵⁴R⁵⁵, CONR⁵⁴OR⁵⁵, NR⁵⁴R⁵⁵, SO₂NR⁵⁴R⁵⁵, S-alkyl, S-alkyl, SOalkyl, SO₂alkyl, Saryl, SOaryl, SO₂aryl; or R⁵² and R⁵³ may be joined to form a cyclic ring (saturated or unsaturated) from 5 to 8 atoms, optionally containing one or more heteroatoms selected from the group N, O or S. R⁵⁴, R⁵⁵, and R⁵⁶ are independently hydrogen, alkyl, alkenyl, alkynyl, perfluoroalkyl, cycloalkyl, cycloheteroalkyl, aryl, substituted aryl, heteroaryl, substituted or hetero alkyl or the like.

“Hetero” when used to describe a compound or a group present on a compound means that one or more carbon atoms in the compound or group have been replaced by a nitrogen, oxygen, or sulfur heteroatom. Hetero may be applied to any of the hydrocarbyl groups described above such as alkyl, e.g. heteroalkyl, cycloalkyl, e.g. cycloheteroalkyl, aryl, e.g. heteroaryl, cycloalkenyl, cycloheteroalkenyl, and the like having from 1 to 5, and especially from 1 to 3 heteroatoms.

“Heteroaryl” refers to a monovalent heteroaromatic group derived by the removal of one hydrogen atom from a single atom of a parent heteroaromatic ring system. Typical heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. Preferably, the heteroaryl group is between 5-15 membered heteroaryl, with 5-10 membered heteroaryl being particularly preferred. Particular heteroaryl groups are those derived from thiophene, pyrrole, benzothiophene, benzofuran, indole, pyridine, quinoline, imidazole, oxazole and pyrazine.

Examples of representative heteroaryls include the following:

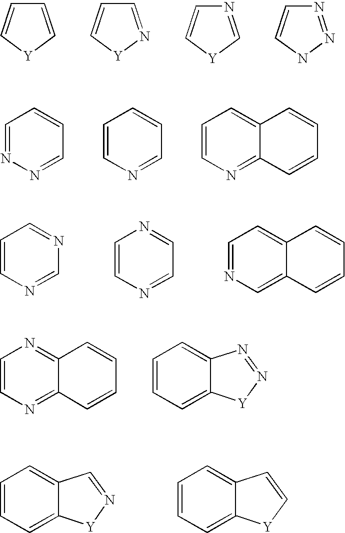

wherein each Y is selected from carbonyl, N, NR⁵⁸, O, and S; and R⁵⁸ is independently hydrogen, alkyl, cycloalkyl, cycloheteroalkyl, aryl, heteroaryl, heteroalkyl or the like.

As used herein, the term “cycloheteroalkyl” refers to a stable heterocyclic non-aromatic ring and fused rings containing one or more heteroatoms independently selected from N, O and S. A fused heterocyclic ring system may include carbocyclic rings and need only include one heterocyclic ring. Examples of heterocyclic rings include, but are not limited to, piperazinyl, homopiperazinyl, piperidinyl and morpholinyl, and are shown in the following illustrative examples:

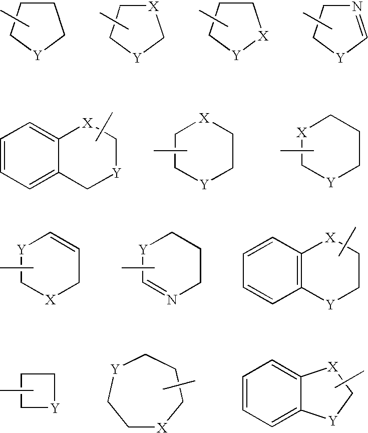

wherein each X is selected from CR⁵⁸, CR⁵⁸₂, NR⁵⁸, O and S; and each Y is selected from NR⁵⁸, O and S; and R⁵⁸ is independently hydrogen, alkyl, cycloalkyl, cycloheteroalkyl, aryl, heteroaryl, heteroalkyl or the like. These cycloheteroalkyl rings may be optionally substituted with one or more groups selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—. Substituting groups include carbonyl or thiocarbonyl which provide, for example, lactam and urea derivatives.

Examples of representative cycloheteroalkenyls include the following:

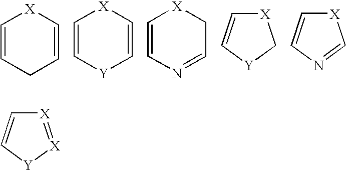

wherein each X is selected from CR⁵⁸, CR⁵⁸₂, NR⁵⁸, O and S; and each Y is selected from carbonyl, N, NR⁵⁸, O and S; and R⁵⁸ is independently hydrogen, alkyl, cycloalkyl, cycloheteroalkyl, aryl, heteroaryl, heteroalkyl or the like.

Examples of representative aryl having hetero atoms containing substitution include the following:

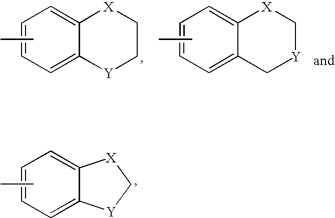

wherein each X is selected from CR⁵⁸, CR⁵⁸₂, NR⁵⁸, O and S; and each Y is selected from carbonyl, NR⁵⁸, O and S; and R⁵⁸ is independently hydrogen, alkyl, cycloalkyl, cycloheteroalkyl, aryl, heteroaryl, heteroalkyl or the like.

“Hetero substituent” refers to a halo, O, S or N atom-containing functionality that may be present as an R⁴ in a R⁴C group present as substituents directly on the ring or rings of the compounds of this invention, or that may be present as a substituent in any “substituted” aryl and aliphatic groups present in the compounds.

Examples of hetero substituents include:
-halo,
—NO₂, —NH₂, —NHR⁵⁹, —N(R⁵⁹)₂,
—NRCOR, —NR⁵⁹SOR⁵⁹, —NR⁵⁹SO₂R⁵⁹, OH, CN,
—CO₂H,
—R⁵⁹—OH, —O—R⁵⁹, —COOR¹⁹,
—CON(R⁵⁹)₂, —CONROR⁵⁹,
—SO₃H, —R⁵⁹, —S, —SO₂N(R⁵⁹)₂, —S(O)R⁵⁹, —S(O)₂R⁵⁹
   wherein each R⁵⁹ is independently an aryl or aliphatic, optionally with substitution. Among hetero substituents containing R⁵⁹ groups, preference is given to those materials having aryl and alkyl R⁵⁹ groups as defined herein. Preferred hetero substituents are those listed above.

“Dihydroxyphosphoryl” refers to the radical —PO(OH)₂.

“Substituted dihydroxyphosphoryl” includes those groups recited in the definition of “substituted” herein, and particularly refers to a dihydroxyphosphoryl radical wherein one or both of the hydroxyl groups are substituted. Suitable substituents are described in detail below.

“Aminohydroxyphosphoryl” refers to the radical —PO(OH)NH₂.

“Substituted aminohydroxyphosphoryl” includes those groups recited in the definition of “substituted” herein, and particularly refers to an aminohydroxyphosphoryl wherein the amino group is substituted with one or two substituents. Suitable substituents are described in detail below. In certain embodiments, the hydroxyl group can also be substituted.

“Thioalkoxy” refers to the group —SR⁶⁰ where R⁶⁰ is alkyl.

“Substituted thioalkoxy” includes those groups recited in the definition of “substituted” herein, and particularly refers to a thioalkoxy group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Sulfanyl” refers to the radical HS—. “Substituted sulfanyl” refers to a radical such as RS— wherein R is any substituent described herein.

“Sulfonyl” refers to the divalent radical —S(O₂)—. “Substituted sulfonyl” refers to a radical such as R⁶¹—(O₂)S— wherein R⁶¹ is any substituent described herein. “Aminosulfonyl” or “Sulfonamide” refers to the radical H₂N(O₂)S—, and “substituted aminosulfonyl” “substituted sulfonamide” refers to a radical such as R⁶²₂N(O₂)S— wherein each R⁶² is independently any substituent described herein.

“Sulfone” refers to the group —SO₂R⁶³. In particular embodiments, R⁶³ is selected from H, lower alkyl, alkyl, aryl and heteroaryl.

“Thioaryloxy” refers to the group —SR⁶⁴ where R⁶⁴ is aryl.

“Thioketo” refers to the group ═S.

“Thiol” refers to the group —SH.

One having ordinary skill in the art of organic synthesis will recognize that the maximum number of heteroatoms in a stable, chemically feasible heterocyclic ring, whether it is aromatic or non aromatic, is determined by the size of the ring, the degree of unsaturation and the valence of the heteroatoms. In general, a heterocyclic ring may have one to four heteroatoms so long as the heteroaromatic ring is chemically feasible and stable.

As used in this specification and the appended claims, the singular forms “a”, “an”, and “the” include plural references unless the context clearly dictates otherwise. Thus for example, reference to “the method” includes one or more methods, and/or steps of the type described herein and/or which will become apparent to those persons skilled in the art upon reading this disclosure.

As used herein, “mammal” refers to any member of the higher vertebrate animals comprising the class Mammalia, which includes, but is not limited to, humans.

For the purposes of this application, the terms “treatment”, “therapeutic use”, and “medicinal use” shall refer to any and all uses of the compositions of the invention which remedy a disease state or one or more symptoms, or otherwise prevent, hinder, retard, or reverse the progression of disease or one or more other undesirable symptoms in any way whatsoever. The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent.

“Pharmaceutically acceptable” means approved by a regulatory agency of the Federal or a state government or listed in the U.S. Pharmacopoeia or other generally recognized pharmacopoeia for use in animals, and more particularly in humans.

“Pharmaceutically acceptable salt” refers to a salt of a compound of the invention that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non toxic organic or inorganic acids, such as hydrochloride, hydrobromide, tartrate, mesylate, acetate, maleate, oxalate and the like.

The term “pharmaceutically acceptable cation” refers to a non toxic, acceptable cationic counter-ion of an acidic functional group. Such cations are exemplified by sodium, potassium, calcium, magnesium, ammonium, tetraalkylammonium cations, and the like.

“Pharmaceutically acceptable vehicle” refers to a diluent, adjuvant, excipient or carrier with which a compound of the invention is administered.

“Preventing” or “prevention” refers to a reduction in risk of acquiring a disease or disorder (i.e., causing at least one of the clinical symptoms of the disease not to develop in a subject that may be exposed to or predisposed to the disease but does not yet experience or display symptoms of the disease).

“Prodrugs” refers to compounds, including derivatives of the compounds of the invention, which have cleavable groups and become by solvolysis or under physiological conditions the compounds of the invention which are pharmaceutically active in vivo. Such examples include, but are not limited to, choline ester derivatives and the like, N-alkylmorpholine esters and the like.

“Solvate” refers to forms of the compound that are associated with a solvent, usually by a solvolysis reaction. Conventional solvents include water, ethanol, acetic acid and the like. The compounds of the invention may be prepared e.g. in crystalline form and may be solvated or hydrated. Suitable solvates include pharmaceutically acceptable solvates, such as hydrates, and further include both stoichiometric solvates and non-stoichiometric solvates.

“Subject” includes humans. The terms “human,” “patient” and “subject” are used interchangeably herein.

“Therapeutically effective amount” means the amount of a compound that, when administered to a subject for treating a disease, is sufficient to effect such treatment for the disease. The “therapeutically effective amount” can vary depending on the compound, the disease and its severity, and the age, weight, etc., of the subject to be treated.

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder, or even preventing the same. In a still further embodiment, “treating” or “treatment” refers to administration of the compound or composition of the invention for cosmetic purposes.

Other derivatives of the compounds of this invention have activity in both their acid and acid derivative forms, but in the acid sensitive form often offers advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well know to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides and anhydrides derived from acidic groups pendant on the compounds of this invention are preferred prodrugs. In some cases it is desirable to prepare double ester type prodrugs such as (acyloxy)alkyl esters or ((alkoxycarbonyl)oxy)alkylesters. Preferred are the C₁ to C₈ alkyl, C₂-C₈ alkenyl, aryl, C₇-C₁₂ substituted aryl, and C₇-C₁₂ arylalkyl esters of the compounds of the invention.

As used herein, the term “isotopic variant” refers to a compound that contains unnatural proportions of isotopes at one or more of the atoms that constitute such compound. For example, an “isotopic variant” of a compound can contain one or more non-radioactive isotopes, such as for example, deuterium (²H or D), carbon-13 (¹³C), nitrogen-15 (¹⁵N), or the like. It will be understood that, in a compound where such isotopic substitution is made, the following atoms, where present, may vary, so that for example, any hydrogen may be ²H/D, any carbon may be ¹³C, or any nitrogen may be ¹⁵N, and that the presence and placement of such atoms may be determined within the skill of the art. Likewise, the invention may include the preparation of isotopic variants with radioisotopes, in the instance for example, where the resulting compounds may be used for drug and/or substrate tissue distribution studies. The radioactive isotopes tritium, i.e. ³H, and carbon-14, i.e. ¹⁴C, are particularly useful for this purpose in view of their ease of incorporation and ready means of detection. Further, compounds may be prepared that aee substituted with positron emitting isotopes, such as ¹¹C, ¹⁸F, ¹⁵O and ¹³N, and would be useful in Positron Emission Topography (PET) studies for examining substrate receptor occupancy.

All isotopic variants of the compounds provided herein, radioactive or not, are intended to be encompassed within the scope of the invention.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

“Tautomers” refer to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base.

Tautomeric forms may be relevant to the attainment of the optimal chemical reactivity and biological activity of a compound of interest.

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

### The Compounds

As described herein, the present invention relates to the identification of compounds that potentiate the activity of antineoplastic agents such as temozolomide and dacarbazine, and more particularly function to sensitize the target cells to the action of these antineoplastic agents. The compounds that have been found to have this activity have been noted to function as antifolate agents, and therefore the invention extends to the combination of antifolate agents and the noted antineoplastic agents, for the treatment of various cancers. To the extent that the compounds and compositions are suited for the treatment of brain cancers such as gliomas and astrocytomas, as well as all other cancers that have metastasized to the brain, such as metastatic melanomas, the inventive antifolate agents are advantageously capable of crossing the blood-brain barrier.

Accordingly, in a first aspect of the invention, a method for treating cancer comprising administering to a cancer patient a combination of an antineoplastic agent and an antifolate agent. In a particular embodiment, the antineoplastic agent may be selected from dacarbazine and temozolomide, and the antifolate agent is capable of crossing the blood-brain barrier. In a further particular embodiment, the antifolate agent is a lipophilic antifolate agent.

In a further aspect of the invention, a method is disclosed for the enhancement or potentiation of the antineoplastic activity of agents such as temozolomide, by the administration of an antifolate agent as defined and disclosed herein. More particularly, the present method may extend to the sensitization of the cells to be treated, to the activity of the antineoplastic agents, by the administration of the present antifolate agents.

The antifolate agent may be of formula I:

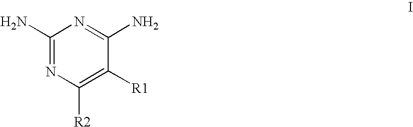

wherein
R¹ is selected from substituted or unsubstituted phenyl;
R² is H, alkyl, substituted alkyl, acyl, substituted acyl, substituted or unsubstituted acylamino, substituted or unsubstituted alkylamino, substituted or unsubstituted alkylthio, substituted or unsubstituted alkoxy, alkoxycarbonyl, substituted alkoxycarbonyl, substituted or unsubstituted alkylarylamino, arylalkyloxy, substituted arylalkyloxy, amino, aryl, substituted aryl, arylalkyl, substituted or unsubstituted sulfoxide, substituted sulfonyl, substituted sulfanyl, substituted or unsubstituted aminosulfonyl, substituted or unsubstituted arylsulfonyl, azido, carboxy, substituted or unsubstituted carbamoyl, cyano, substituted or unsubstituted cycloalkyl, substituted or unsubstituted cycloheteroalkyl, substituted or unsubstituted dialkylamino, halo, heteroaryloxy, substituted or unsubstituted heteroaryl, substituted or unsubstituted heteroalkyl, hydroxy, nitro, and thiol;
or a pharmaceutically acceptable salt thereof; and stereoisomers and tautomers thereof.

In a further embodiment of the invention, the antifolate agent is of formula II:

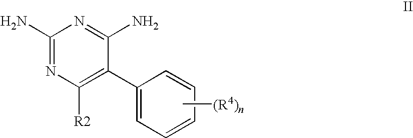

wherein
R² is selected from H, substituted or unsubstituted alkyl; substituted or unsubstituted alkoxy, halo, or CN;
R⁴ is H, alkyl, substituted alkyl, acyl, substituted acyl, substituted or unsubstituted acylamino, substituted or unsubstituted alkylamino, substituted or unsubstituted alkylthio, substituted or unsubstituted alkoxy, alkoxycarbonyl, substituted alkoxycarbonyl, substituted or unsubstituted alkylarylamino, arylalkyloxy, substituted arylalkyloxy, amino, aryl, substituted aryl, arylalkyl, substituted or unsubstituted sulfoxide, substituted sulfonyl, substituted sulfanyl, substituted or unsubstituted aminosulfonyl, substituted or unsubstituted arylsulfonyl, azido, carboxy, substituted or unsubstituted carbamoyl, cyano, substituted or unsubstituted cycloalkyl, substituted or unsubstituted cycloheteroalkyl, substituted or unsubstituted dialkylamino, halo, heteroaryloxy, substituted or unsubstituted heteroaryl, substituted or unsubstituted heteroalkyl, hydroxy, nitro, and thiol; n is 1, 2, 3, 4, or 5;
or a pharmaceutically acceptable salt thereof; and stereoisomers and tautomers thereof.

In a more particular embodiment with respect to compounds of formula II, R² is selected from H, substituted or unsubstituted alkyl; substituted or unsubstituted alkoxy, halo, or CN; R⁴ is H, substituted or unsubstituted alkyl; substituted or unsubstituted alkoxy, halo, or CN; and n is 1, 2, 3, 4, or 5.

In a further particular embodiment, the antifolate agent is of formula II; and each R⁴ is H.

In a further particular embodiment, the antifolate agent is of formula II; and n is 1; and R⁴ is substituted or unsubstituted alkoxy.

In a further particular embodiment, the antifolate agent is of formula II; and n is 1; and R⁴ is OMe, OEt, O-i-Pr, or O-n-Bu.

In a further particular embodiment, the antifolate agent is of formula II, R⁴ and n are as stated above; and R² is H. In a further particular embodiment, R² is substituted or unsubstituted alkyl. In a further particular embodiment, R² is Me, Et, n-Pr, i-Pr, i-Bu or n-Bu.

In a further particular embodiment, the antifolate agent is selected from pyrimethamine, trimetrexate, Piritrexim, etoprine, metoprine, cycloguanil, methotrexate, trimethoprim, triamterene, amiloride, aminopterin, N,N-dimethylamiloride, N,N-hexamethyleneamiloride, and pterin-6-carboxylic acid. In a further particular embodiment, the antifolate agent is pyrimethamine.

In certain aspects, the present invention provides prodrugs and derivatives of the compounds of the invention. Prodrugs are derivatives of the compounds of the invention, which have metabolically cleavable groups and become by solvolysis or under physiological conditions the compounds of the invention, which are pharmaceutically active, in vivo. Such examples include, but are not limited to, choline ester derivatives and the like, N-alkylmorpholine esters and the like.

Other derivatives of the compounds of this invention have activity in both their acid and acid derivative forms, but the acid sensitive form often offers advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well know to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides and anhydrides derived from acidic groups pendant on the compounds of this invention are preferred prodrugs. In some cases it is desirable to prepare double ester type prodrugs such as (acyloxy)alkyl esters or ((alkoxycarbonyl)oxy)alkylesters. Preferred are the C₁ to C₈ alkyl, C₂-C₈ alkenyl, aryl, C₇-C₁₂ substituted aryl, and C₇-C₁₂ arylalkyl esters of the compounds of the invention.

The present invention also relates to the pharmaceutically acceptable acid addition and base salts of any of the aforementioned compounds of formulae I and II. The acids which are used to prepare the pharmaceutically acceptable acid addition salts of the aforementioned base compounds of this invention are those which form non-toxic acid addition salts, ie., salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate (i.e., 1,1-methylene-bis-(2-hydroxy-3-naphthoate)) salts.

The compounds useful according to the invention that are basic in nature are capable of forming a wide variety of different salts with various inorganic and organic acids. Although such salts must be pharmaceutically acceptable for administration to animals, it is often desirable in practice to initially isolate a compound of formula I from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the active base compounds of this invention are readily prepared by treating the base compound with a substantially equivalent amount of the chosen mineral or organic acid in an aqueous solvent medium or in a suitable organic solvent, such as methanol or ethanol. Upon careful evaporation of the solvent, the desired solid salt is readily obtained.

The appropriate dose regimen, the amount of each dose administered, and specific intervals between doses of the active compound will depend upon the particular active compound employed, the condition of the patient being treated, and the nature and severity of the disorder or condition being treated. Preferably, the active compound is administered in an amount and at an interval that results in the desired treatment of or improvement in the disorder or condition being treated.

Although methods and materials similar or equivalent to those described herein can be used in the practice or testing of the invention, preferred methods and materials are described below. The materials, methods, and examples are illustrative only and not intended to be limiting. Other features and advantages of the invention will be apparent from the detailed description, examples, and the claims.

### Pharmaceutical Compositions

Because in vivo use is contemplated, the compositions are preferably of high purity and substantially free of potentially harmful contaminants, e.g., at least National Food (NF) grade, generally at least analytical grade, and preferably at least pharmaceutical grade. To the extent that a given compound must be synthesized prior to use, such synthesis or subsequent purification shall preferably result in a product that is substantially free of any potentially contaminating toxic agents that may have been used during the synthesis or purification procedures.

In preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 70 percent active ingredient. Suitable solid carriers are known in the art, e.g., magnesium carbonate, magnesium stearate, talc, sugar, lactose. Tablets, powders, cachets and capsules can be used as solid dosage forms suitable for oral administration.

For oral administration, Gelatin capsules or liquid-filled soft gelatin capsules can contain the active ingredient and powdered or liquid carriers, such as lactose, lecithin starch, cellulose derivatives, magnesium stearate, stearic acid, and the like. Similar diluents can be used to make compressed tablets. Both tablets and capsules can be manufactured as sustained release products to provide for continuous release of medication over a period of hours. Compressed tablets can be sugar-coated or film-coated to mask any unpleasant taste and to protect the tablet from the atmosphere, or enteric-coated for selective, targeted disintegration in the gastrointestinal tract. Liquid dosage forms for oral administration can contain coloring and/or flavoring to increase patient acceptance.

For preparing suppositories, a low melting wax such as a mixture of fatty acid glycerides or cocoa butter is first melted, and the active ingredient is dispersed homogeneously therein as by stirring. The molten homogeneous mixture is then poured into convenient sized molds, allowed to cool and thereby solidify.

Liquid form preparations include solutions, suspensions and emulsions. As an example may be mentioned water or water-propylene glycol solutions for parenteral injection.

Also included are solid form preparations which are intended to be converted, shortly before use, to liquid form preparations for either oral or parenteral administration. Such liquid forms include solutions, suspensions and emulsions.

In general, sterile water, oil, saline, aqueous dextrose (glucose), polysorbate and related sugar solutions and glycols such as propylene glycol or polyethylene glycols, are suitable carriers for parenteral solutions. Solutions or emulsions for parenteral administration preferably contain about 5-15% polysorbate 80 or lecithin, suitable stabilizing agents and, if necessary, buffer substances. Antioxidizing agents such as, but not limited to, sodium bisulfite, sodium sulfite, or ascorbic acid, either alone or combined, are suitable stabilizing agents. Also useful are citric acid and its salts, and sodium EDTA. In addition, parenteral solutions can contain preservatives including, but not limited to, benzalkonium chloride, methyl- or propyl-paraben, and chlorobutanol.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

Preferably, the pharmaceutical preparation is in unit dosage form. In such form, the preparation is subdivided into unit doses containing appropriate quantities of the active component, e.g., an effective amount to achieve the desired purpose.

The quantity of active compound in a unit dose of preparation may be varied or adjusted from about 0.1 mg to 1000 mg, more preferably from about 1 mg to 500 mg, according to the particular application.

The actual dosage employed may be varied depending upon the requirements of the patient and the severity of the condition being treated. Determination of the proper dosage for a particular situation is within the skill of the art. For convenience, the total daily dosage may be divided and administered in portions during the day, if desired. The antineoplastic agent may be administered using conventional techniques such as those described in Wasserman et al., Cancer, 36: 1258-1268 (1975). Where appropriate, oral administration at a rate of 40-400 mgm⁻² per day, and preferably 150-300 mgm⁻² per day, in 1-5, and preferably 4-5 doses, over 1-5, and preferably 4-5, consecutive days is highly preferred. Intravenous administration at a daily dose of 25-250 mgm⁻² is preferable for a continuous dosing therapy regimen. Oral administration can be utilized for a repeat dosing regimen.

The antifolate agent can be administered separately prior to, or concurrent with, the antineoplastic agent. Where it is desirable to do so, both the antifolate agent and the antineoplastic agent can be combined into a unit dosage form to facilitate patient dosing. Such combination dosage forms may be in any of the above-described dosage forms, but, as noted above, are preferably in oral or intravenous forms.

The antineoplastic agent and the antifolate agent can be packaged in a kit form. In such a kit, the antineoplastic agent and the antifolate agent would be individually formulated into particular dosage forms for the particular route of administration, and contain instructions for the administration of the contents. In a typical embodiment for oral formulation, such a kit may be in the form of a blister package with separately formulated oral dosage forms of the antineoplastic agent and the antifolate agent.

Any necessary adjustment in dose can be readily made to meet the chemotherapeutic treatment requirements of the individual patient and adjusted accordingly by the skilled practitioner.

The following formulation examples illustrate representative pharmaceutical compositions that may be prepared in accordance with this invention. The present invention, however, is not limited to the following pharmaceutical compositions.

### Formulation 1

### Tablets

A compound of the invention is admixed as a dry powder with a dry gelatin binder in an approximate 1:2 weight ratio. A minor amount of magnesium stearate is added as a lubricant. The mixture is formed into 240-270 mg tablets (80-90 mg of active compound per tablet) in a tablet press.

### Formulation 2

### Capsules

A compound of the invention is admixed as a dry powder with a starch diluent in an approximate 1:1 weight ratio. The mixture is filled into 250 mg capsules (125 mg of active compound per capsule).

### Formulation 3

### Liquid

A compound of the invention (125 mg) may be admixed with sucrose (1.75 g) and xanthan gum (4 mg) and the resultant mixture may be blended, passed through a No. 10 mesh U.S. sieve, and then mixed with a previously made solution of microcrystalline cellulose and sodium carboxymethyl cellulose (11:89, 50 mg) in water. Sodium benzoate (10 mg), flavor, and color are diluted with water and added with stirring. Sufficient water may then added to produce a total volume of 5 mL.

### Formulation 4

### Tablets

A compound of the invention may be admixed as a dry powder with a dry gelatin binder in an approximate 1:2 weight ratio. A minor amount of magnesium stearate is added as a lubricant. The mixture is formed into 450-900 mg tablets (150-300 mg of active compound) in a tablet press.

### Formulation 5

### Injection

A compound of the invention is dissolved or suspended in a buffered sterile saline injectable aqueous medium to a concentration of approximately 5 mg/mL.

### Formulation 6

### Topical

Stearyl alcohol (250 g) and a white petrolatum (250 g) are melted at about 75° C. and then a mixture of a compound of the invention (50 g) methylparaben (0.25 g), propylparaben (0.15 g), sodium lauryl sulfate (10 g), and propylene glycol (120 g) dissolved in water (about 370 g) is added and the resulting mixture is stirred until it congeals.

### General Synthetic Procedures

The compounds of this invention which comprise various known drugs or drug like molecules can be purchased from commercial sources and tested for their activities. The steroidal compounds which are not commercially available can be prepared from readily available starting materials using various general methods and procedures known in the art.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

The following examples illustrate and demonstrate the preparation and use of the compositions and corresponding methods of the invention, and are presented for purposes of illustration and not limitation.

In the series of experiments that follow and that are described in this example, further detailed examination of the antifolate agents identified by the invention, was performed. Many of the experiments that are described below, correspond in procedure to those set forth above, and from the resultant data obtained, both expand and validate the observations that the antifolate agents of the invention exert a favorable effect on the activity of the antineoplastic agents with which they are administered.

### Example 1

### Screening of Spectrum Library in Melanoma Cells

### Tumor Cell Lines:

Human malignant melanoma cell lines (SK-MEL19, SK-MEL100, SK-MEL173, and SK-MEL192) were a gift of Dr. Alan Houghton (Memorial Sloan-Kettering Cancer Center, New York, N.Y.) and glioma cell lines (T98-G and LN-18) were obtained from the American Type Culture Collection in Rockville, Md. Cells were cultured in Dulbecco's modified Eagle's medium (DMEM) supplemented with 10% fetal calf serum at 37° C.

### Spectrum Library:

The library used in this study was The Spectrum Collection (MicroSource Discovery Inc., Gaylordsville, Conn. 06755). The 2000 compounds in this library are either marketed drugs, other biologically active small molecules, or natural products (supplied at a concentration of 10 mM in dimethyl sulfoxide (DMSO)).

### Drugs and Reagents:

TMZ (NSC 362856) and the lipophilic folate analogs metoprine (DDMP), etoprine (DDEP), trimetrexate, Piritrexim and cycloguanil (CYC) were kindly provided by National Cancer Institute (Bethesda, Md.). Pyrimethamine, methotrexate (MTX), and leucovorin was purchased from Sigma-Aldrich Chemical Company, Inc. (St. Louis, Mo.). TMZ was dissolved in DMSO in a 4 mg/ml stock solution. PYR was dissolved in DMSO in a 10 mM stock and stored at −20° C. before use.

Identification of Compounds that Enhance TMZ Chemotherapeutic Efficacy by Screening the Spectrum Collection Library

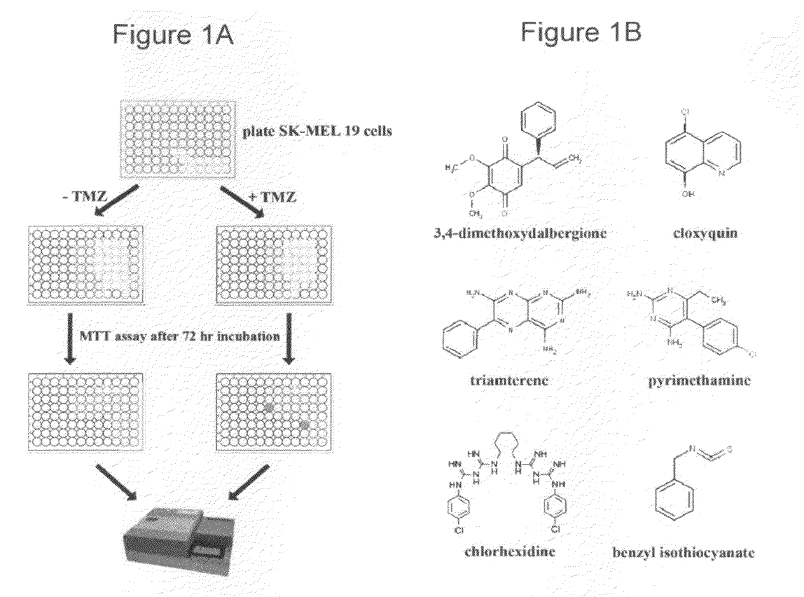

A commercial library of 2000 drugs and natural products was screened to identify novel compounds that enhance TMZ-induced growth inhibition in the human melanoma cell line SK-MEL 19. Screening was performed in 96-well plates at final compound concentration of 1 μM, in the absence or presence of 50 μg/ml TMZ, followed by MTT assay (FIG. 1A). Six compounds (benzyl isothiocyanate, chlorhexidine, cloxyquin, 3,4-dimethoxydalbergione, pyrimethamine and triamterene) were identified as potent enhancers with a minimum 50% greater growth inhibition relative to TMZ alone (FIG. 1B). These positive candidates were reconfirmed by testing their activity at a lower final concentration of 0.5 μM. PYR was chosen for further study due to its long-established and safe use in human as an antimalarial drug.

### Statistical Analysis

With regard to the data presented in the examples, such data were expressed as means±standard deviation. Statistical analysis was performed using the Student's Paired t-test. The criterion for statistical significance was established as a probability value <0.05.

### Example 2

### Cell Proliferation Assay and Viability Assay

The cell growth and cytotoxicity after treatment with TMZ and/or PYR on melanoma cells and glioma cells was performed using Colorimetric MTT assay for cell survival and proliferation assay (MTT Assay Kit, Promega). Tumor cells were seeded at 3000 cells per well (100 μl volume) in 96-well plates and allowed to attach overnight. After TMZ and PYR treatment for 72 hours, the cells were stained with MTT, which is then converted to dark blue formazan crystals by mitochondrial dehydrogenases in viable cells. The plates were read with a microplate reader by measuring the absorbance of converted MTT at 490 nm. Results were expressed as the OD₄₉₀ relative to that of untreated cells. Cell viability was also determined by the trypan-blue dye exclusion method.

### Results:

### PYR Enhanced the Chemotherapeutic Efficacy of TMZ Through Cell Proliferation and Survival Inhibition

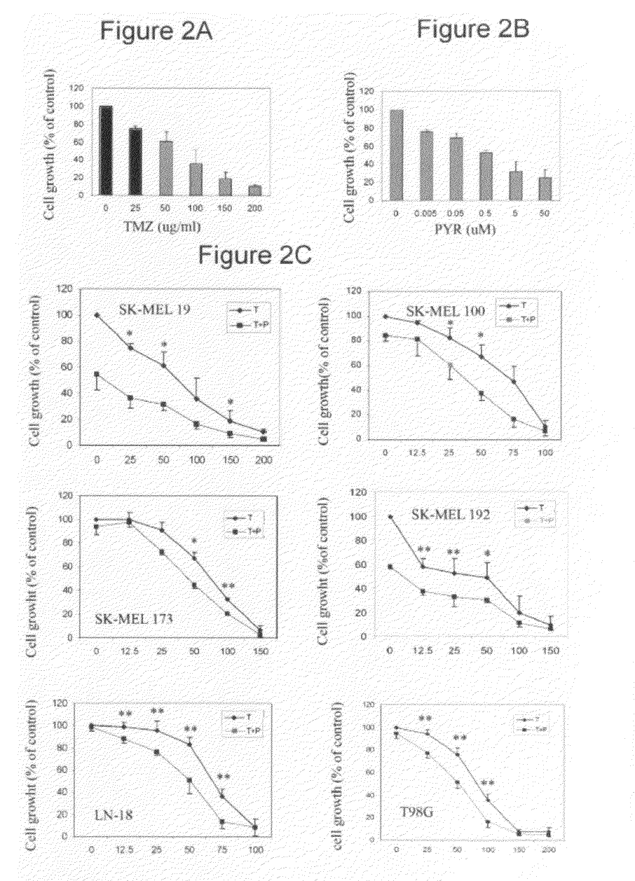

The effect of either TMZ alone, PYR alone or their combination treatment (TMZ/PYR) on melanoma cell proliferation was examined in this example (FIG. 2). TMZ and PYR alone exhibited a dose-dependent inhibition of SK-MEL 19 cell proliferation (FIGS. 2A, 2B). Unless otherwise noted, 25 μg/ml TMZ and 0.5 μM PYR were used in subsequent experiments based on response curves and clinically relevant concentrations.

The effect of the TMZ/PYR combination treatment was further examined by treating three additional melanoma cell lines (SK-MEL 100, 173 and 192) and two glioma cell lines (LN-18 and T98G), with either TMZ alone or the TMZ/PYR combination. Treatment with TMZ/PYR resulted in a significant decrease in cell proliferation (P<0.05, student t-test) in all six cell lines tested in this study (FIG. 2C).

Next, the effect of PYR on TMZ was examined in a panel of melanoma cells and glioma cells. A similar sensitization is also observed in melanoma cells SKM173, SKM192, and glioma cell T98G. The results are set forth in FIGS. 2A-2C, and demonstrate that the combination of the two agents has broad potential clinical utility.

### Example 3

### Cell Cycle Analysis

Cell cycle distribution was determined by staining DNA with propidium iodide (PI, Sigma). Briefly, cells were treated with TMZ, PYR or both for 72 h and then harvested. Cells were then washed and fixed in 70% ethanol on ice for 30 min. After centrifugation, the cell pellets were washed and resuspended in phosphate-citrate buffer. Cells were then treated with ribonuclease and stained with propidium iodide. DNA content was analyzed on a cytofluorimeter by fluorescence-activated cell sorting analysis (FACScan).

### PYR Altered TMZ-Induced Cell Cycle Arrest

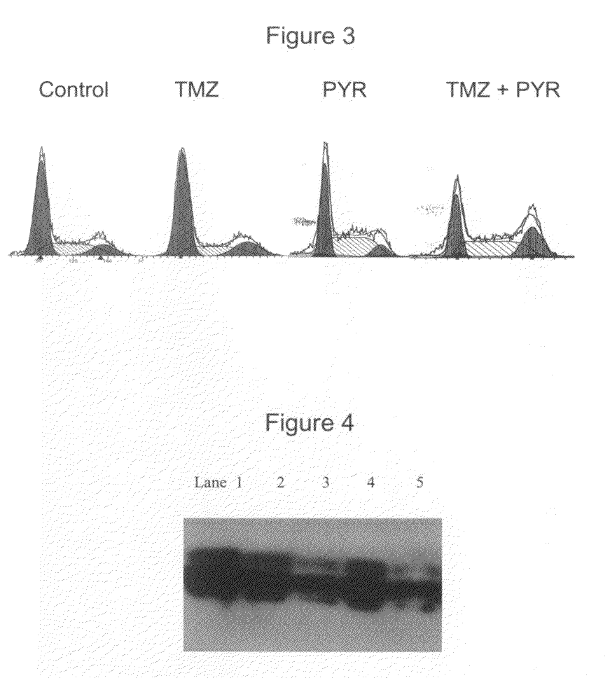

In order to address whether the anti-proliferative effect of TMZ/PYR combination in melanoma cells is associated with cell cycle regulation, DNA cell cycle analysis was performed with cells treated with TMZ, PYR, or TMZ/PYR. No obvious changes were observed in cells treated with TMZ at a concentration of 25 μg/ml (FIG. 3). PYR at a final concentration of 0.5 μM increased the number of S phase cells (FIG. 3). More dramatic accumulation of cells in the S phase accompanied by a decrease in the proportion in G0/G1 was observed in the cells treated with the combination of both compounds on Day 3 (FIG. 3). Cell accumulation in S-phase following PYR and TMZ/PYR treatment suggests that a significant proportion of cells were unable to complete DNA synthesis correctly and cell division was interrupted.

The concentration of TMZ (25 μg/ml or ˜100 μM) used in this study was relatively low and close to clinically achievable serum levels during chemotherapy (100 μM; ref. 23). The data suggests that at 25 μg/ml TMZ (˜100 μM) has no effect on cell cycle progression in melanoma cells in this system.

### Results:

The combined use of the antifolate compound pyrimethamine (PYR) and TMZ sensitized cancer cells to TMZ. In comparison to treatment with TMZ alone, combination treatment increased the inhibition of cell proliferation, enhanced cell cycle arrest, increased DNA damage and apoptosis. The increase in cell death due to combination treatment could be rescued by leucovorin, a reduced form of folate. Other folate antagonists were also found to be effective enhancers of TMZ-induced cytotoxicity.

In this experiment, the activity of PYR as an antifolate was explored by the testing of a group of agents that are known to possess that activity. Accordingly, tests were conducted with methotrexate (MTX), trimethoprim (TMP), trimetrexate (TMTX), pyritrexim (PTX) and cycloguanil (CYC). The results are set forth in FIG. 3 and demonstrate that the members of the group tested all favorably inhibit cell growth.

### Example 4

Drug-induced folate deficiency is associated with accumulation of cells in S-phase accumulation, gene instability, DNA damage and apoptosis. To further understand the mechanism by which PYR enhances TMZ efficacy, the apoptotic response to PYR, TMZ and the combination of the two, was analyzed. Cytotoxic chemotherapy can trigger cancer cell death by activating an apoptotic cascade. Caspase-3 is one of the key “executioners” of apoptosis. The presence of activated caspase-3 in treated cells was assessed by immunoblotting with an antibody specific to the cleaved form of caspase-3. It was found that cleaved caspase-3 was increased in cells treated with both TMZ and PYR compared to those treated with either single agent. Drug resistance in melanoma has also been partially attributed to overexpression of Bcl-2, an anti-apoptotic protein. In an experiment depicted in FIG. 4, it was discovered that Bcl-2 is preferentially down regulated in cells treated with the combination of TMZ and PYR compared to treatment with either agent alone.

### Example 5

### PYR Enhanced Cell Death and Apoptosis Induced by TMZ in Melanoma Cells

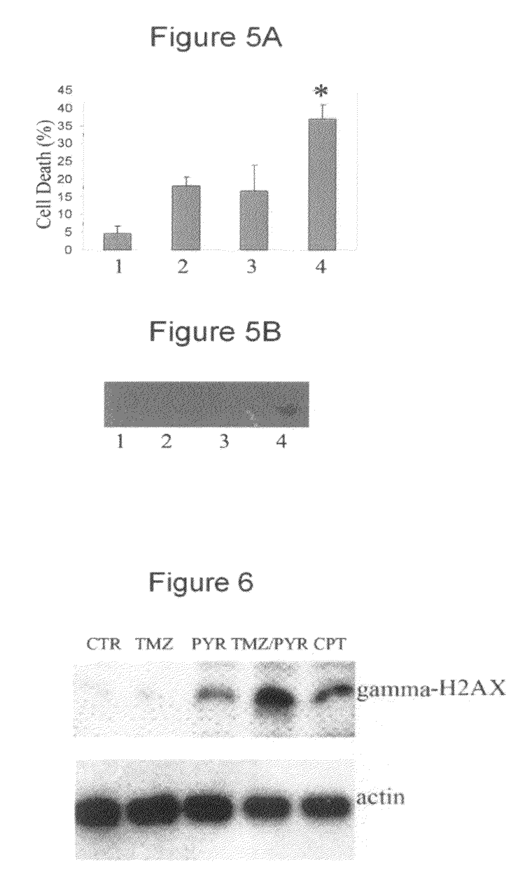

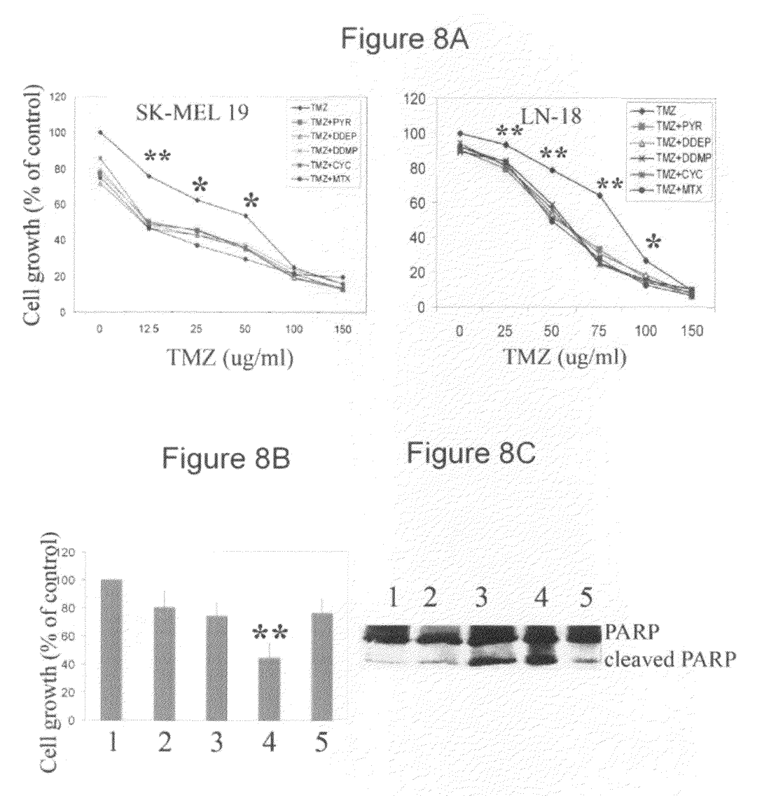

To investigate whether PYR can enhance the induction of cell death by TMZ, Trypan blue staining was employed to determine the ratio of dead cells versus total cells in SK-MEL 19. At clinically-relevant concentrations, TMZ induced death in approximately 15% of cells (FIG. 5A), while PYR at 0.5 μM had only a limited effect on cell death. However, there was an approximately 2 fold increase in cell death when SK-MEL 19 was treated with the combination of TMZ/PYR (FIG. 5A). These results are consistent with the MTT assay data (FIG. 8C, infra.) and suggest that the PYR/TMZ combination treatment significantly enhances the inhibition of melanoma cell growth and induction of cell death by TMZ.

### Western Blot Analysis:

Cells were harvested in extraction buffer (1% Triton X-100, 50 mM Tris, 2 mM EDTA, 150 mM NaCl, pH 7.5) containing complete protease inhibitor mixture (Roche, Mannheim, Germany). The lysates were centrifuged at 14,000 rpm for 10 min at 4° C. Bio-Rad protein assay reagent (Bio-Rad, Philadelphia, Pa.) was used to measure the protein concentrations. Proteins (20 μg) were separated by 8, 12 or 15% sodium dodecyl sulfate-polyacrylamide (SDS-PAGE) gel and transferred to Immobilon-P membranes (Millipore).

In order to determine whether TMZ, PYR or TMZ/PYR induce cell death by the same pathway, cellular levels of activated caspase-3 was assessed by immunoblot analysis with an antibody against the cleaved (active) form of the enzyme. No obvious change was observed in cells treated with either compound, while the level of activated caspase-3 increased significantly when SK-MEL 19 cells were treated with TMZ/PYR (FIG. 5B, FIG. 8C). The results suggest that the proapoptotic effects of TMZ/PYR combination treatment in melanoma cells are mediated by a caspase-dependent pathway.

### Example 6

In this experiment, the effect of treatment on DNA damage was investigated. Certain of the materials and methods involved, are set forth below.

### Antibodies:

Anti-phospho-H2A.X (Ser 139) antibody was purchased from Upstate Cell Signaling Solutions (Temecula, Calif.), anti-O⁶-methylguanine-DNA-methyltransferase (MGMT) antibody was from Chemicon (Danvers, Mass.), and antibodies against capase-3, PARP, β-actin were from Cell Signaling Technology (Beverly, Mass.).

### Results:

### TMZ/PYR Treatment Increased DNA Damage in Melanoma Cells

DNA damage is a well-characterized initial, upstream event in apoptotic cell death (24). Phosphorylation of histone H2AX is one of the earliest responses to strand breakage and is accepted as an early marker for DNA double strand breaks DSBs (25). We used the phosphorylated histone H2AX to determine whether TMZ/PYR combination treatment caused more DSBs. The topoisomerase inhibitor camptothecin, which induces DSB by stalling DNA replication forks, was used as positive control, while untreated cells served as negative control. A trace level of phosphorylated histone H2AX was observed in both negative control cells and cells treated with TMZ alone (FIG. 6). PYR treatment induced a modest increase in the level of phosphorylated histone H2AX, indicating that PYR induces cell death through its activity as an antifolate. A significant increase in the level of phosphorylated histone H2AX was observed in cells treated with TMZ/PYR (FIG. 6). These results demonstrated that TMZ/PYR combination treatment generates more DSBs, which further induces cell death.

Because the O⁶-meG lesion induced by TMZ treatment can be directly removed by the DNA repair protein O⁶-methylguanine-DNA-methyltransferase (MGMT), the cellular level of MGMT is one of the main contributors to TMZ resistance (26). Western blot analysis was performed to determine whether TMZ, PYR, and TMZ/PYR treatments could alter MGMT protein levels. No alteration in MGMT protein level was observed in cells treated with either agent alone or in combination (data not shown), suggesting that the additive antiproliferative effect exerted by the TMZ/PYR combination is independent of effects on MGMT levels.

### Example 7

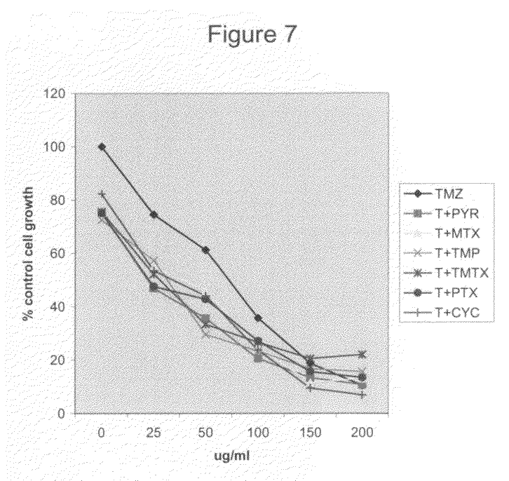

Among the six “hits” identified from library screening, both PYR and triamterene are antifolate compounds. To investigate whether the compounds in this group inhibit cell growth, a representative group including methotrexate (MTX), trimethoprim (TMP), tremetrexate ((TMTX), pyretrexim (PTX) and cycloguanil (CYC) were all tested in combination with TMZ to determine whether they inhance TMZ chemotherapeutic efficacy through their activity as antifolates. The results are set forth in FIG. 7, and demonstrate that the tested compounds all favorably inhibited cell growth.

### Example 8

### PYR Antifolate Activity Enhances TMZ Chemotherapeutic Efficacy in Melanoma and Glioma Cells

In this example, further tests of additional antifolate compounds were performed to determine whether they, too, enhanced TMZ-induced growth inhibition in both SK-MEL-19 and LN-18 cell lines. Similar effects of cell growth inhibition were observed with all antifolates studied. For example, the IC₅₀ of TMZ was reduced from ˜60 μg/ml to ˜12 μg/ml in SK-MEL-19 cells when co-treated with each of five different antifolates (DDEP, DDMP, CYC, MTX and PYR, FIG. 8A).

Leucovorin (N-5-formyltetrahydrofolate, LV, folinic acid), a reduced form of folate, is used widely to specifically reverse the toxic effects of antifolates (27). To further confirm that PYR-enhanced TMZ chemotherapeutic efficacy in melanoma cells was due to the antifolate activity of PYR, 10 μM LV was added to cells treated with TMZ/PYR. LV rescued both the inhibition of cell growth (FIG. 8B) and the increase in apoptotic death induced by TMZ/PYR as measured by the proteolytic cleavage of PARP by caspase-3 (FIG. 8C). These results further confirmed that PYR enhances TMZ chemotherapeutic efficacy through its function as an antifolate, most likely by virtue of its action as a competitive inhibitor of DHFR.

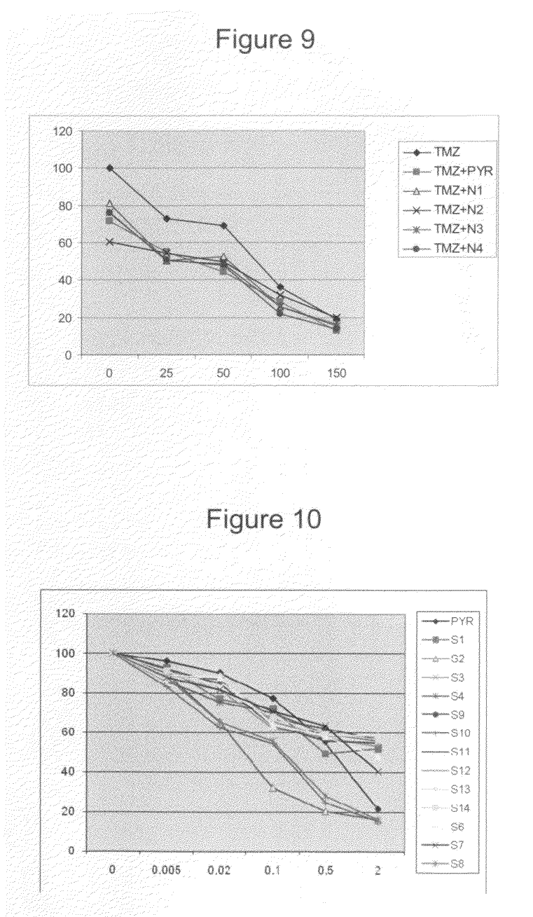

In FIGS. 9 and 10, additional antifolate agents were tested, and the results indicating their activity as enhancers of TMZ activity are set forth.

### Discussion

In the current study, we employed a chemical genetic approach to identify agents to overcome TMZ resistance in melanoma. The commercially available Spectrum library was screened using a chemotherapy-resistant melanoma cell line to identify compounds that enhance TMZ chemotherapeutic efficacy. The advantages of this library are three-fold. First, many compounds in the library have been used for other indications in humans, therefore facilitating clinical testing. Secondly, based on the known function and mechanism of action of these compounds, one can readily test whether the chemosensitization effect of these agents are carried out via the same mechanism. Finally, after identification of a “hit” compound, additional compounds in the same class can be tested, providing useful information regarding structure-activity relationships.

Our screen of the Spectrum library was the first to identify pyrimethamine (PYR), a lipophic DHFR inhibitor with clinical efficacy as an antimalarial drug, as a compound that significantly improved TMZ efficacy in both melanoma and gliomas. In addition, PYR also enhanced cell killing induced by DTIC, a metabolite of TMZ that acts via the same mechanism (data not shown). However, the fact that other antimalarial compounds could not mimic the effect of pyrimethamine on enhancing the TMZ or DTIC chemotherapeutic efficacy in melanoma cells indicates that PYR does not enhance TMZ chemotherapeutic efficacy through its activity as an antimalarial compound (data not shown). Instead, further characterization unexpectedly and unobviously revealed that PYR enhanced TMZ efficacy through its activity as an antifolate compound.

Many attempts have been made to enhance TMZ efficacy through combination treatments (7-12). The other agents used in combination with TMZ interfered with DNA repair pathways through either MGMT-inhibitor O⁶-benzylguanine (28) or base excision repair inhibitor methoxyamine (29, 30). Alternatively, TMZ has been combined with cytotoxic agents, radiotherapy, immunotherapy or anti-angiogenic agents in clinical trials, but compared with the effect of TMZ alone, no clear benefits have been demonstrated from these combinations (31).

Previous studies have shown that TMZ at high concentration can induce cell death and apoptosis in both melanoma and glioma cells (30, 31) and TMZ induces G2 arrest in glioma cells (32). However, a recent report suggests that TMZ induces senescence but not apoptosis in human melanoma cells (35). In our study, at a clinically relevant concentration (˜25 μg/ml), TMZ alone has little effect on apoptosis, DNA damage, and cell cycle arrest in melanoma cells. These results reflect the very modest clinical performance of TMZ as a chemotherapeutic agent. The TMZ/PYR combination described in this study is novel in that we have identified that folate metabolism is involved in TMZ sensitization in melanoma and glioma cells. Furthermore, the effective concentrations of both TMZ and PYR are clinically achievable, which differs significantly from some previously used agents.

PYR is known as a DHFR inhibitor, particularly in protozoa such as malaria and toxoplasma. DHFR inhibitors have been studied for many years as antineoplastic agents. The disruption of folic acid metabolism has long been known to inhibit cell growth. Folic acid is essential for the de novo synthesis of the nucleoside thymidine, which is required for DNA synthesis. Thus, antifolates have greater toxicity on rapidly dividing cells such as tumor cells. Several PYR analogues, such as etoprine (DDEP) and metoprine (DDMP), have been investigated as antitumor agents (36). It has been reported that folate deficiency can induce apoptosis and S phase accumulation in various cell lines (37-39). Consistent with previous reports (39), we observed that PYR at concentration of 0.5 μM increased the number of cells in S phase (FIG. 3).

It has been reported that folate deficiency in cell culture induces an excess of strand breaks in DNA (40, 41) and preliminary data from a human study also indicate that genome-wide DNA strand breaks are related to folate status (41). Folate deficiency induces DNA damage such as stand breakage due to either decreased thymidylate synthesis (43) or an altered dNTP pool (44). In this study, we observed both S phase accumulation (FIG. 3) and an increase in DSBs (FIG. 6) in cells treated with PYR/TMZ compared to each agent alone. These results suggest that PYR enhances TMZ chemotherapeutic efficacy through its antifolate activity. We further confirmed this mechanism of action by showing that other antifolates also enhanced the chemotherapeutic efficacy of TMZ in melanoma cells and that the effects of antifolates could be rescued by the leucovorin (folinic acid). Our findings reveal that targeting folate metabolism, especially through DHFR inhibition, is an effective strategy to improve TMZ efficacy in melanoma.

O⁶-methylguanine-DNA-methyltransferase (MGMT) plays crucial roles in the repair of DNA damage induced by TMZ treatment because it directly removes O⁶meG (25). The expression level of MGMT is also susceptible to epigenetic silencing (45). In our study, we showed that TMZ/PYR treatment induced more DNA damage through the formation of DSBs (FIG. 6). However, no changes in MGMT protein level were observed in cells treated with PYR, TMZ or PYR/TMZ (data not shown), suggesting that, in melanoma, the enhancement of TMZ efficacy by PYR is independent of MGMT level. This observation was further confirmed by the finding that PYR-induced sensitization of cells to TMZ treatment was observed in both MGMT expressing and MGMT negative cells, which finding will be clinically important (FIG. 2).

There are many other pathways that are involved in repairing DNA damage induced by methylating agents, including base excision repair (BER), homologus recombination, polymerase bypass, and mismatch repair (MMR). For example, previous studies have indicated that inhibition of MGMT increased TMZ sensitivity only in MMR-proficient, but not in MMR-deficient cells (46). DNA repair is a complex process in that the major pathway and proteins involved in DNA repair is cell-type dependent (47). In this study, it was found that MGMT is not a target of PYR-induced TMZ sensitization.

This study has identified the unexpected utility of PYR and of antifolates, as chemotherapy enhancers, in both melanoma and glioma cells. PYR is bioavailable following oral administration and has also been safely administered to malarial patients and those with other protozoal infections such as toxoplasmosis for prolonged periods with limited side effects. In addition, PYR differs from the classical antifolates such as MTX in that it is a lipophilic antifolate, allowing it to diffuse readily across the cell membrane without a transporter. Furthermore, PYR easily crosses the blood-brain barrier. All of these taken together make PYR an attractive candidate for clinical efficacy and safety testing both in melanoma (including, importantly, melanoma metastatic to the brain) and glioma.

In the data presented above, it has been shown that PYR can greatly enhance the chemotherapeutic efficacy of TMZ and that the combination of TMZ/PYR resulted in significantly decreased cell growth in human melanoma and glioma cells. Moreover, PYR and the combination of TMZ/PYR caused an accumulation of cells in S phase and subsequent cell cycle arrest. Increases in DSBs and apoptosis were also observed with PYR/TMZ treatment. Overall, it has been demonstrated herein that the TMZ/PYR combination treatment induced DNA damage, cell cycle arrest, apoptosis, and reduced cellular proliferation.

Moreover, PYR and the combination of TMZ/PYR caused an accumulation of cells in S phase and subsequent cell cycle arrest. Increases in DSBs and apoptosis were also observed with PYR/TMZ treatment. Overall, it has been demonstrated herein that the TMZ/PYR combination treatment induced DNA damage, cell cycle arrest, apoptosis, and reduced cellular proliferation.

As stated above, many of the antifolate agents already demonstrate safety in clinical use, and in the instance of pyrimethamine, its oral availability (which matches up with temozolomide) combined with the fact that like temozolomide, it can effectively cross the blood-brain barrier. This is especially important if it were to be used to in conjunction with temzolomide to treat melanoma with brain metastatses or brain cancers like glioma and glioblastoma.

### REFERENCES

1. Atkins M B. The treatment of metastatic melanoma with chemotherapy and biologics. Curr Opin Oncol 9: 205-213, 1997
2. Balch C M, Soong S J, Gershenwald J E et al: Prognostic factors analysis of 17,600 melanoma patients: validation of the American Joint Committee on Cancer Melanoma Staging System. J Clin Oncol 19: 3622-3634, 2001
3. Markovic S N, Erickson L A, Rao R D, Weenig R H, Pockaj B A, Bardia A B, et al: Malignant melanoma in the 21^{st} century, part 2: Staging, Prognosis, and Treatment. Mayo Clin Proc 82(4): 490-513, 2007
4. Wolchok J D and Saenger Y M: Current topics in melanoma. Curr Opin Oncol 19: 116-120, 2007
5. Mutter N, et al (2006): TMZ: a milestone in neuro-oncology and beyond? Expert Rev. Anticancer Ther 6(8): 1187-1204
6. Weller M (2005): TMZ, a milestone in the pharmacotherapy of brain tumors. Future Oncol 1(6): 747-754
7. Bafaloukos D, Gogas H, Briassoulis E et al: Temozolomide and docetaxel as first line chemotherapy in patients with advanced melanoma. A phase II study of the Hellenic Cooperative Oncology Group. Proc Am Soc Clin Oncol 19: 543A, 2000
8. Atkins M B, Gollob J A, Sosman J A et al: A phase II pilot trial of concurrent biochemotherapy with cisplatin, vinblastin, temozolomide, interleukin, and IFN-α2B in patients with metastatic melanoma. Clin Cancer Res 8: 3075-3081, 2002
9. Hwu W J, Krown S E, Menell J H, et al: Phase II study of temozolomide plus thalidomide for the treatment of metastatic melanoma. J Clin Oncol 21: 3351-3356, 2003
10. Silvani A, Eoli M, Salmaggi A, Lamperti E, Maccagnano E, Broggi G and Boiardi A: Phase II trial of cisplatin plus temozolomide, in recurrent and progressive malignant glioma patients. J Neurooncol 66: 203-208, 2004
11. Gogas H, Polyzos A, Stavrinidis I, Frangia K, Tsoutsos D, Panagiotou P, Markopoulos C, Papadopoulos O, Pectasides D, Mantzourani M, Middleton M, Vaiopoulos G and Fountzilas G: Temozolomide in combination with celecoxib in patients with advanced melanoma. A phase II study of the Hellenic Cooperative Oncology Group. Annals of Oncology 17: 1835-1841, 2006
12. Fountzilas G, Karkavelas G, Kalogera-Fountzila A, Karina M, Ignatiadis M, Koukoulis G, Plataniotis G, Misailidou D, Bobos M, Pectasides D, Razis E, Karavelis A and Selviaridis P: Post-operative combined radiation and chemotherapy with temozolomide and irinotecan in patients with high-grade astrocytic tumors. A phase II study with biomarker evaluation. Anticancer Reserch 26: 4675-4686, 2006
13. Stockwell B R. Chemical genetics: ligand-based discovery of gene function. Nat Rev Genet 1: 116-125, 2000
14. Tan D S. Sweet surrender to chemical genetics. Nat Biotechnol 2002; 20, 561-563
15. Schreiber S L. The small-molecule approach to biology. Chem Eng News 2003; 81, 51-61
16. Khersonsky S M, Chang Y T. Strategies for facilitated forward chemical genetics. Chem-biochemistry 2004; 5, 903-908
17. Smukste I, Stockwell B R. Advances in chemical genetics. Annu Rev Genomics Hum Genet 2005; 6, 262-286
18. Zon L I, Peterson R T. In vivo drug discovery in the zebrafish. Nat Rev 2005, 4, 35-44
19. Williams D, Jung D W, Khersonsky S, Heidary N, Chang Y T, Orlow S J. Identification of compounds that bind mitochondrial F1F0 ATPase by screening a triazine library for correction of albinism. Chem Biol 2004; 11, 1251-1259
20. Jung D W, Williams D, Khersonsky S M, Kang T W, Heidary N, Chang Y T et al. Identification of the F1F0 mitochondrial ATPase as a target for modulating skin pigmentation by screening a tagged triazine library in zebrafish. Mol BioSyst 2005, 1, 85-92
21. Ni-Komatsu L, Leung J K, Williams D, Min J, Khersonsky S M, Chang Y T and Orlow S J: Triazine-based tyrosinase inhibitors identified by chemical genetic screening. Pigment Cell Res 18: 447-453, 2005
22. Snyder J R, Hall A, Ni-Komatsu L, Khersonsky S M, Chang Y T, Orlow S J. Dissection of melanogenesis with small molecules identifies prohibition as a regulator. Chem Biol 2005; 12, 477-484
23. Hammond L A, Eckardt J R, Kuhn J G, Gerson S L, Johnson T, Smith L et al: A randomized phase I and pharmacological trial of sequences of 1,3-bis(2-chloroethyl)-1-nitrosourea and temozolomide in patients with advanced solid neoplasms. Clin Cancer Res 10: 1645-1656, 2004
24. Lips J and Kaina B: DNA double-strand breaks trigger apoptosis in p53-deficient fibroblasts. Carcinogenesis 22: 579-585, 2001
25. Jackson S P. Sensing and repairing DNA double-strand breaks. Carcinogenesis 2002; 23:687-96
26. Gerson S L: Clinical relevance of MGMT in the treatment of cancer. J Clin Oncol 20: 2388-2399, 2002
27. Keshava C, Keshava N, Whong W Z, Nath J, Ong T M: Inhibition of methotrexate-induced chromosomal damage by folinic acid in V79 cells. Mutat Res 397:221-228, 1998
28. Dolan M E and Pegg A E: O⁶-benzylguanine and its role in chemotherapy. Clin Cancer Res 3: 837-847, 1997
29. Fishel M L, He Y, Smith M L, Kelley M R. Manipulation of base excision repair to sensitize ovarian cancer cells to alkylating agent temozolomide. Clin Can Res 2007; 13, 260-267
30. Yan L, Bulgar A, Miao Y, et al. Combined treatment with temozolomide and methoxyamine: blocking apurininc/pyrimidinic site repair coupled with targeting topoisomerase IIalpha. Clin Cancer Res 2007; 13: 1532-9.
31. Quirt I, Verma S, Petrella T, Bak K, Charette M. Temozolomide for the treatment of metastatic melanoma: A systematic review. The Oncologist 2007; 12: 1114-23.
32. D'Atri S, Tentori L, Lacal P M, Graziani G, Pagani E, Benincase E, Zambruno G, Bonmassar E and Jirieny J: Involvement of the mismatch repair system in temozolomide-induced apoptosis. J Mol Pharmacol 54: 334-341, 1998
33. Roos W P, Batista L F, Naumann S C, Wick W, Weller M, Menck C F and Kaina B: Apoptosis in malignant glioma cells triggered by the temozolomide-induced DNA lesion O⁶-methylguanine. Oncogene 26(2): 186-197, 2007
34. Hirose Y, Berger M S, Pieper R O: Abrogation of the Chk1-mediated G(2) checkpoint pathway potentiates temozolomide-induced toxicity in a p53-independent manner in human glioblastoma cells. Cancer Res 61: 5843-5849, 2001
35. Mhaidat N M, Zhang X D, Allen J, Avery-Kiejda K A, Scott R J, Hersey P. Temozolomide induces senescence but not apoptosis in human melanoma cells. Br J Cancer 2007; 97: 1225-33.
36. Murphy M L, Ellison R R, Karnofsky D A and Burchenal J H: Clinical effects of the dichloro and mono-chlorophenyl analogues of diamino pyrimidine: antagonists of folic acid. J Clin Invest 33: 1388-1396, 1954
37. James S J, Pogribny I P, Pogribna M, Miller B J, Jernigan S, and Melnyk S: Mechanisms of DNA damage, DNA hypomethylation, and tumor progression in the folate/methyl-deficient rat model of hepatocarcinogenesis. J Nutr 133: S3740-S3737, 2003
38. Lin H, Chen C, Tsai W, Yen J and Liu H: In vitro folate deficiency induces apoptosis by a p53, Fas (Apo-1, CD95) independent, bcl-2 related mechanism in phytohaemagglutinin-stimulated human peripheral blood lymphocytes. British Journal of Nutrition 95: 870-878, 2006
39. Huang R F, Ho Y H, Lin H L, Wei J S and Liu T Z: Folate deficiency induces a cell cycle-specific apoptosis in HepG2 cells. J Nutr 129: 25-31, 1999
40. Branda R F and Blickensderfer D B: Folate deficiency increases genetic damage caused by alkylating agents and γ-irradiation in Chinese hamster ovary cells. Cancer Res 53: 5401-5408, 1993
41. Courtemanche C, Huang A C, Elson-Schwab I, Kerry N, Ng B Y, and Ames B N: folate deficiency and ionizing radiation cause DNA breaks in primary human lymphocytes: a comparison. FASEB J 18: 209-211, 2004
42. Lathrop-Stern L, Selhub J, Choi S W and Mason J B: Genomic DNA stand breaks in peripheral leukocytes are correlated with RBC folate concentrations in human subjects. FASEB J 14: A294 (abs.), 2000
43. Duthie S J and Hawdon A: DNA instability (strand breakage, uracil misincorporation, and defective repair) is increased by folic acid depletion in human lymphocytes in vitro. FASEB J 12, 1491-1497, 1998
44. Yoshioka A, Tanaka S, Hiraoka O, Koyama Y, Ayusawa D, Seno T, Garrett C, Wataya Y. Deoxyribonucleoside triphosphate imbalances. 5-Fluorodeoxyuridine-induced DNA double strand breaks in mouse FM3A cells and mechanism of cell death. J Bio Chem 1987; 262, 8235-8241
45. Donson A M, Addo-Yobo S O, Handler M H, Gore L, Foreman N K. MGMT promoter methylation correlates with survival benefit and sensitivity to temozolomide in pediatric glioblastoma. Pediatr Blood Cancer 2007; 48, 403-407
46. Alvino E, Castiglia D, Caporali S, et al. A single cycle of treatment with temozolomide, alone or combined with O6-benzylguanine, induces strong chemoresistance in melanoma cell clones in vitro: role of O6-methylguanine-DNA methyltransferase and the mismatch repair system. Int J Oncol 2006; 29: 785-97
47. Waytt M D and Pittman D L: Methylating agents and DNA repair responses: methylated base and sources of strand breaks. Chem Res Toxicol 19: 1580-1594, 2006

While certain of the preferred embodiments of the present invention have been described and specifically exemplified above, it is not intended that the invention be limited to such embodiments. Various modifications may be made thereto without departing from the scope and spirit of the present invention, as set forth in the following claims.

From the foregoing description, various modifications and changes in the compositions and methods of this invention will occur to those skilled in the art. All such modifications coming within the scope of the appended claims are intended to be included therein.

All publications, including but not limited to patents and patent applications, cited in this specification are herein incorporated by reference as if each individual publication were specifically and individually indicated to be incorporated by reference herein as though fully set forth.

The chemical names of compounds given in this application were generated using various commercially available chemical naming software tools including MDL's ISIS Draw Autonom Software tool, and were not verified. Particularly, in the event of inconsistency, the depicted structure governs.

1. A method for treating cancer comprising administering to a cancer patient a combination of temozolomide and an antifolate agent, wherein the antifolate agent is capable of crossing the blood-brain barrier.

2. A method for increasing the efficacy of temozolomide in the treatment of cancer comprising administering to a patient an effective amount of temozolomide in combination with an antifolate agent, wherein the antifolate agent is capable of crossing the blood-brain barrier.

3. (canceled)

4. A method for treating advanced cancer sensitive to the combination below in patients in need of such treatment comprising administering temozolomide and an antifolate agent, wherein the antifolate agent is capable of crossing the blood-brain barrier.

5. The method of claim 1 wherein the cancer may be found in the brain.

6. The method of claim 5 wherein the cancer is a brain cancer.

7. The method of claim 5 wherein the cancer is, or is capable of, metastasizing to the brain.

8. The method of claim 1 wherein the antifolate agent is a lipophilic antifolate agent.

9. A method of claim 1 wherein the antifolate agent is of formula I: 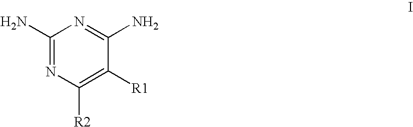
wherein
R¹ is selected from substituted or unsubstituted phenyl;
R² is H, alkyl, substituted alkyl, acyl, substituted acyl, substituted or unsubstituted acylamino, substituted or unsubstituted alkylamino, substituted or unsubstituted alkylthio, substituted or unsubstituted alkoxy, alkoxycarbonyl, substituted alkoxycarbonyl, substituted or unsubstituted alkylarylamino, arylalkyloxy, substituted arylalkyloxy, amino, aryl, substituted aryl, arylalkyl, substituted or unsubstituted sulfoxide, substituted sulfonyl, substituted sulfanyl, substituted or unsubstituted aminosulfonyl, substituted or unsubstituted arylsulfonyl, azido, carboxy, substituted or unsubstituted carbamoyl, cyano, substituted or unsubstituted cycloalkyl, substituted or unsubstituted cycloheteroalkyl, substituted or unsubstituted dialkylamino, halo, heteroaryloxy, substituted or unsubstituted heteroaryl, substituted or unsubstituted heteroalkyl, hydroxy, nitro, and thiol;
or a pharmaceutically acceptable salt thereof;
and stereoisomers and tautomers thereof.

10. A method of claim 1, wherein the antifolate agent is of formula II: 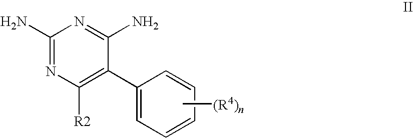
wherein
R² is selected from H, substituted or unsubstituted alkyl; substituted or unsubstituted alkoxy, halo, or CN;
R⁴ is H, alkyl, substituted alkyl, acyl, substituted acyl, substituted or unsubstituted acylamino, substituted or unsubstituted alkylamino, substituted or unsubstituted alkylthio, substituted or unsubstituted alkoxy, alkoxycarbonyl, substituted alkoxycarbonyl, substituted or unsubstituted alkylarylamino, arylalkyloxy, substituted arylalkyloxy, amino, aryl, substituted aryl, arylalkyl, substituted or unsubstituted sulfoxide, substituted sulfonyl, substituted sulfanyl, substituted or unsubstituted aminosulfonyl, substituted or unsubstituted arylsulfonyl, azido, carboxy, substituted or unsubstituted carbamoyl, cyano, substituted or unsubstituted cycloalkyl, substituted or unsubstituted cycloheteroalkyl, substituted or unsubstituted dialkylamino, halo, heteroaryloxy, substituted or unsubstituted heteroaryl, substituted or unsubstituted heteroalkyl, hydroxy, nitro, and thiol; n is 1, 2, 3, 4, or 5;
or a pharmaceutically acceptable salt thereof;
and stereoisomers and tautomers thereof.

11. A method of claim 10 wherein the antifolate agent is of formula II; and wherein R² is selected from H, substituted or unsubstituted alkyl; substituted or unsubstituted alkoxy, halo, or CN;
R⁴ is H, substituted or unsubstituted alkyl; substituted or unsubstituted alkoxy, halo, or CN; and n is 1, 2, 3, 4, or 5.

12. A method of claim 10, wherein the antifolate agent is of formula II; and wherein each R⁴ is H.

13. A method of claim 10, wherein the antifolate agent is of formula II; and wherein n is 1; and R⁴ is substituted or unsubstituted alkoxy.

14. A method of claim 10, wherein the antifolate agent is of formula II; and wherein n is 1; and R⁴ is OMe, OEt, O-i-Pr, or O-n-Bu.

15. A method of claim 10, wherein the antifolate agent is according to claim 10; and R² is H.

16. A method of claim 10, wherein the antifolate agent is according to claim 10; and R² is substituted or unsubstituted alkyl.

17. A method of claim 10, wherein the antifolate agent is according to claim 10; and R² is Me, Et, n-Pr, i-Pr, i-Bu or n-Bu.

18. A method of claim 1 wherein the antifolate agent is selected from pyrimethamine, trimetrexate, Piritrexim, etoprine, metoprine, cycloguanil, methotrexate, trimethoprim, triamterene, amiloride, aminopterin, N,N-dimethylamiloride, N,N-hexamethyleneamiloride, and pterin-6-carboxylic acid.

19. A method of claim 1 wherein the antifolate agent is pyrimethamine trimetrexate, or Piritrexim.

20. A method of claim 1 wherein the antifolate agent is etoprine, methoprine, or methotrexate.

21. A method of claim 1 wherein the antifolate agent is cycloguanil, or trimethoprim.

22. A method of claim 1 wherein the antifolate agent is pyrimethamine.

23. A method of claim 1 wherein the antifolate agent is triamterene.

24. A method of claim 1 wherein the antifolate agent is amiloride, N,N-dimethylamiloride, or N,N-hexamethyleneamiloride.

25. A method of claim 1 wherein the antifolate agent is aminopterin, or pterin-6-carboxylic acid.

26. The method of claim 1, wherein the dosage administered of said antifolate agent is about 1-2000 mg/kg of patient body weight.

27. The method of claim 1, wherein the dosage administered of the antifolate agent is 10-800 mg/kg of patient body weight.

28. The method of claim 27, wherein the temozolomide is administered at a rate of 150-300 mgm^{′2} of body surface area per day.

29. The method of claim 1, wherein said antifolate agent and said temozolomide are administered in divided doses on consecutive days.

30. The method of claim 1, wherein said antifolate agent is administered in a dose of 10-800 mg/kg of patient body weight prior to the administration of the temozolomide, said temozolomide is administered in an amount of 150-300 mgm″² of body surface area per day, and said antifolate agent and said temozolomide are administered in divided doses on consecutive days.

31. The method of claim 30, wherein the antifolate agent selected from pyrimethamine, trimetrexate, Piritrexim, etoprine, metoprine, cycloguanil, methotrexate, trimethoprim, triamterene, amiloride, aminopterin, N,N-dimethylamiloride, N,N-hexamethyleneamiloride, and pterin-6-carboxylic acid.

32. The method of claim 31, wherein the total dose of temozolomide is divided into at least four individual doses which are administered on at least four consecutive days.

33. The method of claim 32, wherein said antifolate agent is administered two to eight hours prior to the administration of said temozolomide.

34. A method according to claim 1, wherein the human cancer cells are breast cancer tumor cells, astrocytoma tumor cells, colorectal tumor cells, melanoma tumor cells, mycosis fungoides tumor cells or glioma tumor cells.

35. (canceled)

36. (canceled)

37. (canceled)

38. (canceled)

39. A pharmaceutical composition, for use in treating human cancer cells in a patient in need of such treatment, comprising an effective amount of an antifolate agent of claim 1, and an effective amount of temozolomide.

40. A kit comprising a pharmaceutical dosage form of temozolomide and a separate pharmaceutical dosage form of an antifolate agent of claim 1, for use in treating human cancer cells in a patient in need of such treatment.

41. A method for the manufacture of a pharmaceutical composition comprising admixing temozolomide and an antifolate agent of claim 1, with one or more pharmaceutically acceptable carriers.

42. A product comprising temozolomide and an antifolate agent of claim 1, as a combined preparation for simultaneous, separate or sequential administration in the treatment of human cancer cells to a patient in need of such treatment.
